(19) United States
(12) Patent Application Publication
(10) Pub. No.: US 2013/0116563 A1
(43) Pub. Date: May 9, 2013

(54) ULTRASONIC DIAGNOSTIC APPARATUS

(71) Applicant: Yoichi OGASAWARA, Nasushiobara-shi (JP)

(72) Inventor: Yoichi OGASAWARA, Nasushiobara-shi (JP)

(21) Appl. No.: 13/662,579

(22) Filed: Oct. 29, 2012

(30) Foreign Application Priority Data
Nov. 7, 2011 (JP) 2011-243782
Sep. 19, 2012 (JP) 2012-205525

(51) Int. Cl. A61B 8/14; A61B 8/08

(52) U.S. Cl. 600/441; 600/440

(57) ABSTRACT

According to one embodiment, a first unit includes a first ultrasonic image generation unit generating a first ultrasonic image at a first processing speed and with a first processing function based on a reception signal, a connection unit detachably connecting the first unit to a second unit, and a connection detection unit detecting connection between the first and second units. The second unit includes a second ultrasonic image generation unit having a second processing speed faster than the first processing speed and a second processing function higher than the first processing function and generating a second ultrasonic image having larger data than the first ultrasonic image based on the reception signal upon detection of the connection.

### CROSS-REFERENCE TO RELATED APPLICATIONS

This application is based upon and claims the benefit of priority from Japanese Patent Applications No. 2011-243782, filed Nov. 7, 2011; and No. 2012-205525, filed Sep. 19, 2012, the entire contents of all of which are incorporated herein by reference.

### FIELD

Embodiments described herein relate generally to an ultrasonic diagnostic apparatus.

### BACKGROUND

Conventional ultrasonic diagnostic apparatuses are roughly classified into a floor-standing type that is used in hospitals and a portable type that is used for home care and sports activities, in disaster sites, and the like. In general, the size of a floor-standing type ultrasonic diagnostic apparatus is larger than that of a portable type ultrasonic diagnostic apparatus. In addition, the floor-type ultrasonic diagnostic apparatus is more expensive than the portable type ultrasonic diagnostic apparatus in accordance with their scales. Furthermore, the performance and function of the floor-standing type ultrasonic diagnostic apparatus are higher than those of the portable type ultrasonic diagnostic apparatus in accordance with their scales and costs. The portable type ultrasonic diagnostic apparatus generally has the basic performance and function of an ultrasonic diagnostic apparatus. Therefore, the performance and function of the portable type ultrasonic diagnostic apparatus are generally limited as compared with those of the floor-standing type ultrasonic diagnostic apparatus.

The following two measures are taken to support ultrasonic diagnoses in two situations, including a situation (to be referred to as a floor-standing situation hereinafter) in which a floor-standing type ultrasonic diagnostic apparatus is used and a situation (to be referred to as a portable situation hereinafter) in which a portable type ultrasonic diagnostic apparatus is used.

The first measure is to purchase ultrasonic diagnostic apparatuses suitable for the two situations. The first measure poses the problem of cost because of the purchase of a plurality of ultrasonic diagnostic apparatuses.

The second measure is to use an ultrasonic diagnostic apparatus, which is suitable for one of the two situations, for the other situation. Using the floor-standing type ultrasonic diagnostic apparatus for a portable situation raises problems associated with the transportation and mobility of the apparatus. Using the portable type ultrasonic diagnostic apparatus for a floor-standing situation raises problems of insufficient performance and function of the apparatus. Mounting a high-function CPU (Central Processing Unit) and GPU (Graphics Processing Unit) in the portable type ultrasonic diagnostic apparatus will eliminate the problem of the insufficient performance and function of the portable type ultrasonic diagnostic apparatus. However, this technique has the following problems. Mounting a high-function CPU and GPU in an apparatus will lead to increases in power consumption, size, and weight of the apparatus in addition to an increase in cost. These problems will reduce the merit of portability of the portable type ultrasonic diagnostic apparatus.

### BRIEF DESCRIPTION OF THE DRAWINGS

FIG. 1 is a block diagram showing the arrangement of an ultrasonic diagnostic apparatus according to the first embodiment;

FIG. 2 is a flowchart showing an example of a procedure for ultrasonic image generation processing according to the first embodiment;

FIG. 3 is a block diagram showing the arrangement of an ultrasonic diagnostic apparatus according to the second embodiment;

FIG. 4 is a flowchart showing an example of a procedure for ultrasonic image generation processing according to the second embodiment;

FIG. 5 is a block diagram showing the arrangement of an ultrasonic diagnostic apparatus according to the third embodiment;

FIG. 6 is a flowchart showing an example of a procedure for ultrasonic image generation processing according to the third embodiment;

FIG. 7 is a block diagram showing the arrangement of an ultrasonic diagnostic apparatus according to the fourth embodiment;

FIG. 8 is a flowchart showing an example of a procedure for ultrasonic image generation processing according to the fourth embodiment;

FIG. 9 is a block diagram showing the arrangement of an ultrasonic diagnostic apparatus according to the fifth embodiment;

FIG. 10 is a flowchart showing an example of a procedure for ultrasonic image generation processing according to the fifth embodiment;

FIG. 11 is a block diagram showing the arrangement of an ultrasonic diagnostic apparatus according to the sixth embodiment; and

FIG. 12 is a block diagram showing the arrangement of an ultrasonic diagnostic apparatus according to the seventh embodiment.

### DETAILED DESCRIPTION

In general, according to one embodiment, an ultrasonic diagnostic apparatus includes a first unit and a second unit detachably connecting to the first unit.

The first unit includes an ultrasonic probe, a transmission/reception unit, a first ultrasonic image generation unit, a connection unit and a connection detection unit. The transmission/reception unit transmits/receives an ultrasonic wave to/from an object through the ultrasonic probe and generates a reception signal. The first ultrasonic image generation unit generates a first ultrasonic image at a first processing speed and with a first processing function based on the reception signal. The connection unit connects the first unit to the second unit. The connection detection unit detects connection between the first unit and the second unit.

The second unit includes a second ultrasonic image generation unit. The second ultrasonic image generation unit is configured at a second processing speed faster than the first processing speed and with a second processing function higher than the first processing function. The second ultrasonic image generation unit generates a second ultrasonic image based on the reception signal upon detection of the connection, wherein the second ultrasonic image having larger data than the first ultrasonic image.

An ultrasonic diagnostic apparatus according to this embodiment will be described below with reference to the accompanying drawing. The same reference numerals denote constituent elements having almost the same arrangements, and a repetitive description will be made only when required.

### First Embodiment

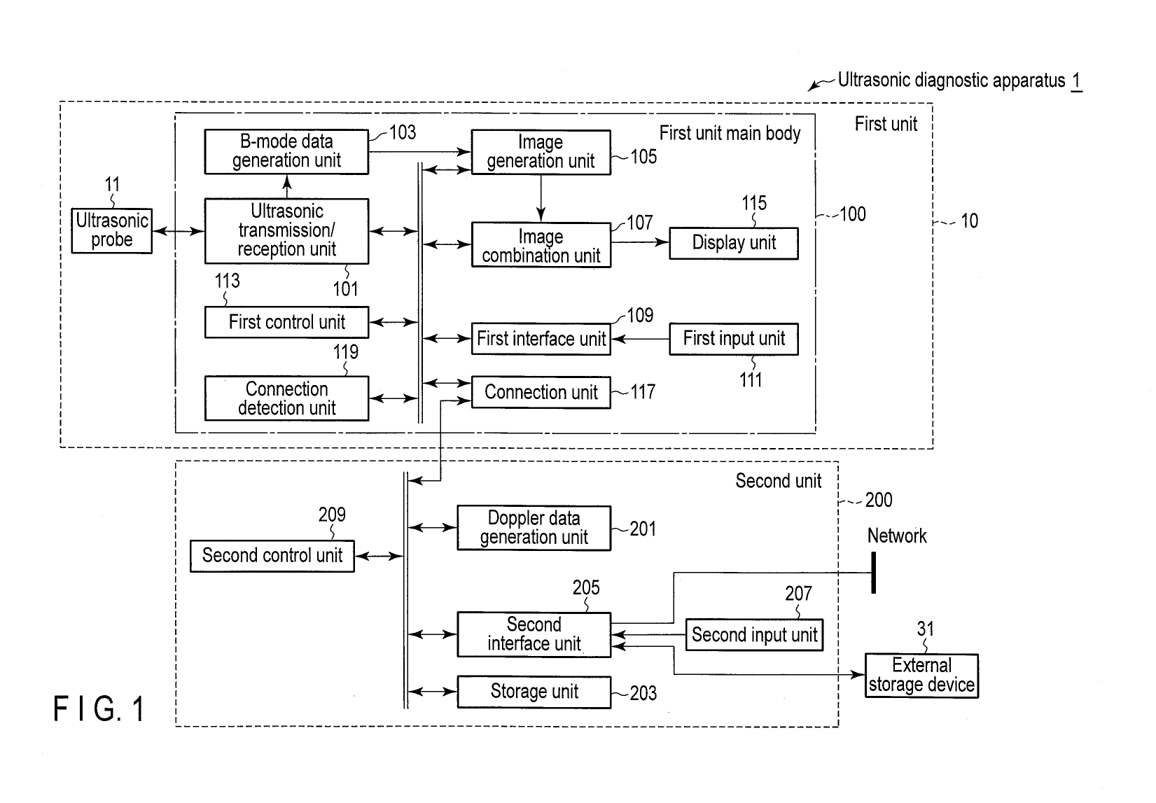

FIG. 1 is a block diagram of an ultrasonic diagnostic apparatus 1 according to the first embodiment. The ultrasonic diagnostic apparatus 1 includes a first unit 10 including an ultrasonic probe 11 and a second unit 200 which is detachable from the first unit 10.

The first unit 10 includes the ultrasonic probe 11 and a first unit main body 100. The first unit main body 100 includes an ultrasonic transmission/reception unit 101, a B-mode data generation unit 103, an image generation unit 105, an image combination unit 107, a first interface unit 109, a first input unit 111, a first control unit 113, a display unit 115, a connection unit 117, and a connection detection unit 119. The first unit 10 has an ultrasonic transmission/reception function and a function of generating a B-mode image. The second unit 200 (to be described later) has a function of generating Doppler data (to be described later). The function of generating Doppler data requires many filters and a complicated calculation function as compared with the function of generating B-mode data (to be described later). For this reason, the first unit 10 is smaller in size and consumes less power than the second unit 200.

The ultrasonic probe 11 includes piezoelectric transducers as reversible acoustic/electric conversion elements such as piezoelectric ceramic elements. A plurality of piezoelectric transducers are juxtaposed and mounted on the distal end of the ultrasonic probe 11. Each piezoelectric transducer generates an ultrasonic wave at a predetermined timing in accordance with a supplied driving signal (voltage pulse). Note that the ultrasonic probe 11 may be a one-dimensional array probe having a plurality of piezoelectric transducers arrayed along one direction or a two-dimensional array probe having a plurality of piezoelectric transducers arrayed in a two-dimensional matrix. Assume that in the following description, one piezoelectric transducer forms one channel.

The ultrasonic transmission/reception unit 101 includes a transmission unit and a reception unit (neither of which is shown). The transmission unit includes a pulse generator, a transmission delay circuit, and a pulser (none of which are shown). The pulse generator repetitively generates rate pulses for the formation of transmission ultrasonic waves at a predetermined rate frequency. The pulse generator repetitively generates rate pulses at a predetermined rate frequency of, for example, 5 kHz. These rate pulses are distributed to channel counts and sent to the transmission delay circuit. The transmission delay circuit gives each rate pulse a delay time necessary to focus an ultrasonic wave into a beam and determine transmission directivity for each channel. Note that a trigger signal generator (not shown) supplies a trigger as a timing signal to the transmission delay circuit. The pulser applies a voltage pulse to each transducer of the ultrasonic probe 11 at the timing when a rate pulse is received from the transmission delay circuit. With this operation, the ultrasonic probe 11 transmits an ultrasonic beam to an object. The number of channels to be driven at the time of ultrasonic transmission can be changed depending on the transmission conditions.

The reception unit includes a preamplifier, analog/digital converter, reception delay circuit, and adder. The preamplifier amplifies an echo signal from the object, which is received via the ultrasonic probe 11, for each channel. The analog/digital converter converts an analog signal into a digital signal. It is possible to change the number of channels used for reception in accordance with the purpose. The reception delay circuit gives the echo signals converted into digital signals delay times required to determine reception directivity. The adder adds a plurality of echo signals in accordance with a reception delay pattern from the first control unit 113 (to be described later). This addition enhances a reflection component from a direction corresponding to the reception directivity. The transmission directivity and the reception directivity determine the comprehensive directivity of ultrasonic transmission/reception (which in turn determines so-called “ultrasonic scanning lines”). Note that the reception unit may have a parallel reception function of simultaneously receiving echo signals generated on a plurality of scanning lines by one ultrasonic transmission.

The ultrasonic transmission/reception unit 101 outputs a generated reception signal to the B-mode data generation unit 103 under the control of the first control unit 113. The ultrasonic transmission/reception unit 101 outputs a generated reception signal to the second unit 200 via the connection unit 117 (to be described later) under the control of a second control unit 209 mounted in the second unit 200 (to be described later).

The B-mode data generation unit 103 includes an envelope detector, logarithmic converter, and analog/digital converter (none of which are shown). The envelope detector performs envelope detection of an input signal from the B-mode data generation unit 103, i.e., the reception signal output from the ultrasonic transmission/reception unit 101. The logarithmic converter relatively enhances a weak signal by logarithmically converting the amplitude of the detected signal. With this operation, the B-mode data generation unit 103 generates B-mode data.

The image generation unit 105 generates a B-mode image based on B-mode data. The image generation unit 105 generates an average velocity image, a variance image, a power image, and a combined image of them based on the Doppler data output from a Doppler data generation unit 201 mounted in the second unit 200 (to be described later). In addition to generating the image, the image generation unit 105 performs conversion (scan conversion) of a scanning line signal string for ultrasonic scanning into a scanning line signal string in a general video format typified by a TV format. The image generation unit 105 generates an ultrasonic image as a display image by this conversion. More specifically, the image generation unit 105 executes the interpolation processing of interpolating data between adjacent scanning lines, enhancement and smoothing processing using various types of filters, frame correlation processing, and the like when executing scan conversion. Note that the image generation unit 105 may include a memory storing image data and execute reconstruction processing of a three-dimensional image and the like.

The image combination unit 107 includes a cine memory and a frame memory (neither of which is shown). The image combination unit 107 combines the image output from the image generation unit 105 with character information of various types of parameters, scale marks, and the like. The image combination unit 107 outputs the combined image to the display unit 115. The cine memory is a memory which stores ultrasonic images corresponding to a plurality of frames immediately before freezing. Continuously displaying (cine displaying) the images stored in this cine memory can display a moving ultrasonic image. The frame memory is a memory which stores an ultrasonic image corresponding to one frame. The display unit 115 displays the image currently stored in the frame memory. Note that the image combination unit 107 can also display a past ultrasonic image of the object stored in a storage unit 203 (to be described later) together with the image generated by the image generation unit 105 while the first unit 10 is connected to the second unit 200.

The first interface unit 109 is an interface associated with the first input unit 111 (to be described later), a biological signal measurement unit (not shown), and the like. The data such as the ultrasonic image and the like obtained by the ultrasonic diagnostic apparatus 1 can be transferred to another apparatus via the first interface unit 109.

The first input unit 111 is connected to the first interface unit 109. The first input unit 111 inputs, to the ultrasonic diagnostic apparatus 1, transmission/reception conditions such as the range of a region of interest (to be referred to as an ROI hereinafter) associated with the B mode, the range scanned with ultrasonic waves (to be referred to as a scanning range hereinafter), a scanning line density, a frame rate, and the number of scanning lines associated with a parallel simultaneous reception function, a selected imaging method, and the like. Imaging methods include, for example, a two-dimensional imaging method, a three-dimensional imaging method, a four-dimensional imaging including time evolution, a tissue harmonic imaging (to be referred to as THI hereinafter) method, and an elastic imaging method.

The first input unit 111 includes input devices such as a trackball, switch buttons, mouse, and keyboard (none of which are shown). The input device detects the coordinates of a cursor displayed on the display screen, and outputs the detected coordinates to the first control unit 113 (to be described later). Note that the input device may be a touch panel provided to cover the display screen. In this case, the first input unit 111 detects a touched and designated coordinates by a coordinate reading principle such as an electromagnetic induction scheme, magnetostriction scheme, or a pressure-sensitive scheme, and outputs the detected coordinates to the first control unit 113. When, for example, the operator operates the end button or freeze button of the first control unit 113, the ultrasonic transmission/reception is terminated, and the ultrasonic diagnostic apparatus 1 is set in a pause state. The first input unit 111 is smaller in size and consumes less power than the input device of a second input unit 207 (to be described later).

The first control unit 113 includes a memory (not shown). The memory stores a plurality of reception delay patterns with different focus depths, control programs for the ultrasonic diagnostic apparatus 1 associated with the B mode, and the like. More specifically, the first control unit 113 reads out a reception delay pattern and a control program stored in the memory based on the transmission/reception conditions input via the first input unit 111 and the selected imaging method. The first control unit 113 controls the first unit 10 of the ultrasonic diagnostic apparatus 1 in accordance with the input transmission/reception conditions and the readout control program.

The first control unit 113 releases the control from each unit mounted in the first unit main body 100 based on the first output from the connection detection unit 119 (to be described later). The first output is information indicating that the first unit 10 is connected to the second unit 200. The first control unit 113 starts controlling each unit mounted in the first unit main body 100 based on the second output from the connection detection unit 119. The second output is information indicating that the first unit 10 is not connected to the second unit 200.

The display unit 115 displays an ultrasonic image based on an output from the image combination unit 107. The display unit 115 includes a liquid crystal display (to be referred to as an LCD hereinafter). Note that the display unit 115 may be a display different from an LCD. For example, the display unit 115 may be arranged near the object instead of being mounted on the first unit 10. At this time, the first unit 10 may be connected to the display unit 115 via the first interface unit 109.

The connection unit 117 connects the first unit 10 to the second unit 200. Note that it is possible to use the first interface unit 109 instead of the connection unit 117.

The connection detection unit 119 detects whether the first unit 10 is connected to the second unit 200 via the connection unit 117. When the first unit 10 is connected to the second unit 200, the connection detection unit 119 outputs information (first output) concerning the connection to the first control unit 113 and the second control unit 209 (to be described later). When the first unit 10 is physically disconnected from the second unit 200, the connection detection unit 119 outputs information (second output) concerning the disconnection to the first control unit 113.

The second unit 200 includes the Doppler data generation unit 201, the storage unit 203, a second interface unit 205, the second input unit 207, and the second control unit 209. Note that a biological signal measurement unit (not shown) typified by an electrocardiograph, phonocardiograph, sphygmograph, or respiration sensor, an external storage device 31, and a network may be connected to the second unit 200 via the second interface unit 205.

The Doppler data generation unit 201 includes a Doppler signal generation unit and a color Doppler data generation unit (neither of which is shown). The Doppler signal generation unit includes a mixer and a low-pass filter (to be referred to as an LPF hereinafter)(neither of which is shown). The mixer multiplies the signal output from the ultrasonic transmission/reception unit 101 of the first unit 10 via the connection unit 117 by a reference signal having a frequency f₀ equal to the transmission frequency. This multiplication will obtain a signal having a component of a Doppler shift frequency f_{d} and a signal having a frequency component of (2f₀+f_{d}). The LPF removes the signal of the high frequency component (2f₀+f_{d}) of the signals having the two types of frequency components from the mixer. The Doppler signal generation unit generates a Doppler signal having the component of the Doppler shift frequency f_{d} by removing the signal of the high frequency component (2f₀+f_{d}). This processing is also called quadrature detection.

The color Doppler data generation unit receives the Doppler signal quadrature-detected by the ultrasonic transmission/reception unit 101 via the connection unit 117 and includes a velocity/variance/power computation unit. The velocity/variance/power computation unit includes an MTI (Moving Target Indicator) filter and an autocorrelation computation unit (neither of which is shown). The MTI filter removes a Doppler component (a clutter component) due to the respiratory movement or pulsatory movement of an organ or the like from the Doppler signal output from the ultrasonic transmission/reception unit 101. The autocorrelation computation unit calculates the autocorrelation value of the Doppler signal obtained by extracting only blood flow information using the MTI filter. The autocorrelation computation unit calculates the average flow velocity value or variance of a blood flow on the basis of the calculated autocorrelation value. The color Doppler data generation unit generates color Doppler data from the average velocity value or variance of blood flows and the like based on a plurality of Doppler signals. The Doppler signal generated by the Doppler signal generation unit and the color Doppler data generated by the color Doppler data generation unit will be collectively referred to as Doppler data hereinafter.

The Doppler data generation unit 201 outputs Doppler data to the image generation unit 105 via the connection unit 117 of the first unit 10.

The storage unit 203 stores the B-mode data generated by the B-mode data generation unit 103, the Doppler data generated by the Doppler data generation unit 201, the ultrasonic image generated by the image generation unit 105, past ultrasonic images of objects, and the like.

The second interface unit 205 is an interface associated with the second input unit 207 (to be described later), a biological signal measurement unit (not shown), the external storage device 31, a network, and the like. It is possible to transfer data such as ultrasonic images obtained by the ultrasonic diagnostic apparatus 1, data stored in the storage unit 203, and the like to the external storage device 31 and other apparatuses via the second interface unit 205.

The second input unit 207 is connected to the second interface unit 205. The second input unit 207 inputs various kinds of instructions, commands, information, selections, and settings from the operator, which are associated with the B mode and the Doppler mode, to the ultrasonic diagnostic apparatus 1. The second input unit 207 includes input devices such as a trackball, switch buttons, mouse, and keyboard (none of which are shown). The input device detects the coordinates of a cursor displayed on the display screen, and outputs the detected coordinates to the second input unit 207. Note that the input device may be a touch panel provided to cover the display screen. In this case, the second input unit 207 detects touched and designated coordinates by a coordinate reading principle such as an electromagnetic induction scheme, magnetostriction scheme, or a pressure-sensitive scheme, and outputs the detected coordinates to the second control unit 209. When, for example, the operator operates the end button or freeze button of the second input unit 207, the ultrasonic transmission/reception is terminated, and the ultrasonic diagnostic apparatus 1 is set in a pause state.

The second input unit 207 and the first input unit 111 have, for example, the following differences. The second input unit 207 inputs various kinds of instructions, commands, information, selections, and settings associate with the B mode and the Doppler mode to the ultrasonic diagnostic apparatus 1. The number of items input via the second input unit 207 is larger than that input by the first input unit 111. For this reason, the second input unit 207 consumes more power than the first input unit 111.

The second control unit 209 includes a memory (not shown). The memory stores a plurality of reception delay patterns with different focus depths, control programs for the ultrasonic diagnostic apparatus 1 associated with the B mode and the Doppler mode, and the like. In response to the first output from the connection detection unit 119, the second control unit 209 controls the first unit 10 and the second unit 200. Upon releasing the control from the first unit 10 in response to the first output and receiving the first output, the second control unit 209 controls the respective units of the first unit 10, except for the first control unit 113, and the respective units of the second unit 200. More specifically, the second control unit 209 reads out a reception delay pattern and a control program stored in the memory based on various kinds of instructions, commands, information, selections, and settings associated with the B mode and Doppler mode which are input via the second input unit 207. The second control unit 209 controls the first unit 10 and the second unit 200 in accordance with the input various kinds of instructions, commands, information, selections, and settings and the readout control program.

The following will describe the processing associated with the generation of ultrasonic images (to be referred to as ultrasonic image generation processing hereinafter) in the ultrasonic diagnostic apparatus 1 which can be separated into the first unit 10 which generates B-mode images and the second unit 200 which generates Doppler data.

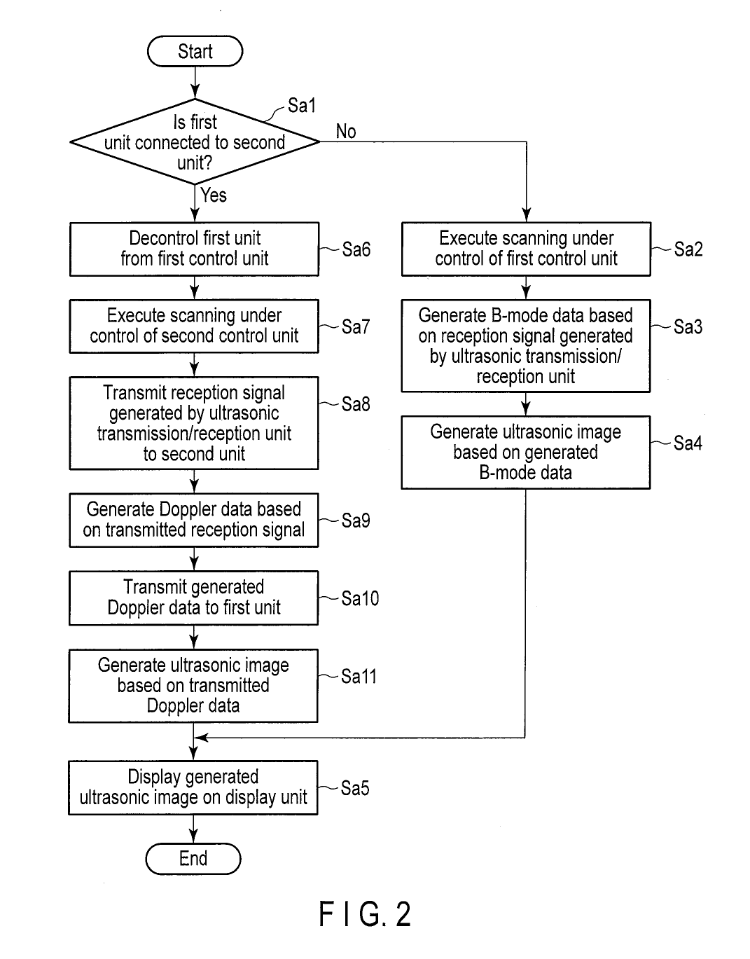

FIG. 2 is a flowchart showing an example of a procedure for ultrasonic image generation processing.

If the first unit 10 is not connected to the second unit 200 via the connection unit 117 (step Sa1), the apparatus executes scanning under the control of the first control unit 113 (step Sa2). The apparatus generates B-mode data based on the reception signal generated by the ultrasonic transmission/reception unit 101 (step Sa3). The apparatus generates an ultrasonic image based on the generated B-mode data (step Sa4). The apparatus displays the generated ultrasonic image on the display unit 115 (step Sa5).

If the first unit 10 is connected to the second unit 200 via the connection unit 117 (step Sa1), the first control unit 113 decontrols the first unit 10 (step Sa6). The apparatus executes scanning under the control of the second control unit 209 (step Sa7). The reception signal generated by the ultrasonic transmission/reception unit 101 is transmitted to the second unit 200 via the connection unit 117 (step Sa8). The apparatus generates Doppler data based on the transmitted reception signal (step Sa9). The generated Doppler data is transmitted to the first unit 10 via the connection unit 117 (step Sa10). The apparatus generates an ultrasonic image based on the transmitted Doppler data (step Sa11).

According to the above arrangement, the following effects can be obtained.

The ultrasonic diagnostic apparatus 1 can be separated into the first unit 10 which generates B-mode data and the second unit 200 which generates Doppler data. This makes it possible to provide an ultrasonic diagnostic apparatus corresponding to each clinical site in a hospital or outside the hospital. That is, separating the first unit 10 from the second unit 200 makes it possible to use the first unit 10 of the ultrasonic diagnostic apparatus 1 as a portable type ultrasonic diagnostic apparatus for home care and sports activities, in disaster sites, and the like. In addition, connecting the first unit 10 to the second unit 200 makes it possible to use the ultrasonic diagnostic apparatus 1 as a high-function floor-standing type ultrasonic diagnostic apparatus in a hospital or the like. In addition, the ultrasonic diagnostic apparatus 1 can lead to a reduction in cost as compared with a case in which both a floor-standing type ultrasonic diagnostic apparatus and a portable type ultrasonic diagnostic apparatus are purchased. As described above, the ultrasonic diagnostic apparatus 1 can provide ultrasonic diagnostic services in accordance with conditions around objects and diagnostic purposes at low cost and with high efficiency.

### Second Embodiment

The second embodiment will be described below with reference to the accompanying drawing.

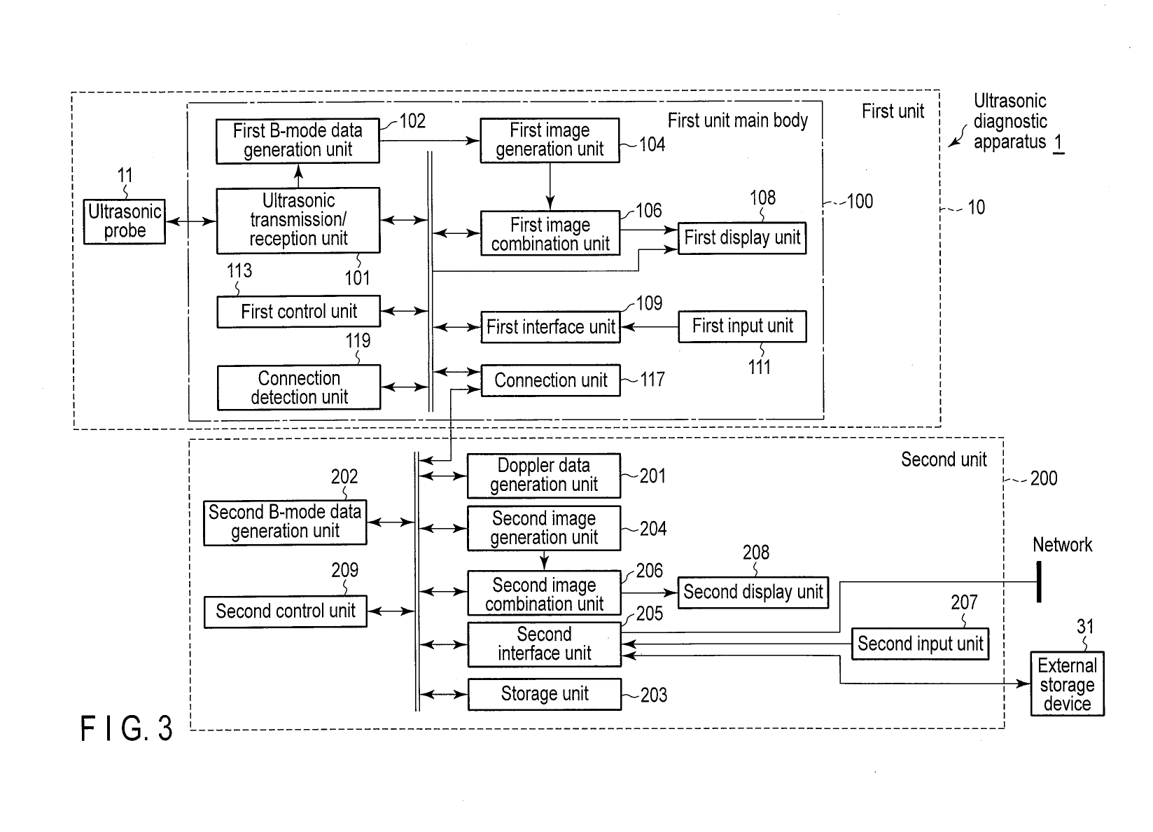

FIG. 3 is a block diagram showing the arrangement of an ultrasonic diagnostic apparatus according to the second embodiment.

The main differences between the second embodiment and the first embodiment are the following four points.

The first point is that the functions associated with the first unit 10 and B-mode data generation unit 103 in the first embodiment are distributed to a first B-mode data generation unit 102 and a second B-mode data generation unit 202. The first B-mode data generation unit 102 is mounted in a first unit 10. The second B-mode data generation unit 202 is mounted in a second unit 200.

The second point is that the function associated with the image generation unit 105 of the first unit 10 in the first embodiment is distributed to a first image generation unit 104 and a second image generation unit 204. The first image generation unit 104 is mounted in the first unit 10. The second image generation unit 204 is mounted in the second unit 200.

The third point is that the function associated with the image combination unit 107 of the first unit 10 in the first embodiment is distributed to a first image combination unit 106 and a second image combination unit 206. The first image combination unit 106 is mounted in the first unit 10. The second image combination unit 206 is mounted in the second unit 200.

The fourth point is that the function associated with the display unit 115 of the first unit 10 in the first embodiment is distributed to a first display unit 108 and a second display unit 208. The first display unit 108 is mounted in the first unit 10. The second display unit 208 is mounted in the second unit 200.

The following will describe those of the constituent elements of the second and first embodiments that operate differently, the first B-mode data generation unit 102, the second B-mode data generation unit 202, the first image generation unit 104, the second image generation unit 204, the first image combination unit 106, the second image combination unit 206, the first display unit 108, and the second display unit 208.

The first unit 10 includes the ultrasonic probe 11 and a first unit main body 100. The first unit main body 100 includes an ultrasonic transmission/reception unit 101, the first B-mode data generation unit 102, the first image generation unit 104, the first image combination unit 106, the first display unit 108, a connection unit 117, a connection detection unit 119, a first control unit 113, a first interface unit 109, and the first input unit 111.

The first input unit 111 is connected to the first interface unit 109. The first input unit 111 inputs, to an ultrasonic diagnostic apparatus 1, at least one of a plurality of transmission/reception conditions such as the number of transmission channels associated with the B mode, the number of reception channels, the range of an ROI, a scanning range, a scanning line density, a frame rate, and the number of scanning lines associated with a parallel simultaneous reception function.

The first control unit 113 includes a memory (not shown). The memory of the first control unit 113 stores a plurality of reception delay patterns with different focus depths, control programs for the first unit main body 100 associated with the B mode, and the like. Upon receiving at least one of a plurality of transmission/reception conditions such as the number of transmission channels, the number of reception channels, the range of an ROI, a scanning range, a scanning line density, a frame rate, and the number of scanning lines associated with a parallel simultaneous reception function, the first control unit 113 determines other transmission/reception conditions based on the data processing ability of the first B-mode data generation unit 102 (to be described later). The first control unit 113 reads out a reception delay pattern and control program stored in the memory based on the determined transmission/reception conditions. The first control unit 113 controls the first unit 10 of the ultrasonic diagnostic apparatus 1 in accordance with the input and determined transmission/reception conditions and the readout control program.

Note that the first control unit 113 may read out a control program from the memory based on the input transmission/reception conditions. At this time, the first control unit 113 decimates reception signals output from the ultrasonic transmission/reception unit 101 so as to match the determined transmission/reception conditions.

The first control unit 113 releases the control from the first B-mode data generation unit 102, first image generation unit 104, and first image combination unit 106 mounted in the first unit 10 based on the first output from the connection detection unit 119. The first output is information indicating that the first unit 10 is connected to the second unit 200. The first control unit 113 starts controlling each unit included in the first unit 10 based on the second output from the connection detection unit 119. The second output is information indicating that the first unit 10 is not connected to the second unit 200.

The ultrasonic transmission/reception unit 101 transmits ultrasonic waves to the object through the ultrasonic probe 11. The ultrasonic transmission/reception unit 101 receives reflected waves corresponding to the transmitted ultrasonic waves from the object. The ultrasonic transmission/reception unit 101 generates a reception signal based on the received reflected waves. The ultrasonic transmission/reception unit 101 outputs the generated reception signal to the first B-mode data generation unit 102 under the control of the first control unit 113. When the first unit 10 is connected to the second unit 200, the ultrasonic transmission/reception unit 101 outputs the generated reception signal to the second unit 200 via the connection unit 117 under the control of the second control unit 209 (to be described later).

The first B-mode data generation unit 102 includes an envelope detector, logarithmic converter, and analog/digital converter (none of which are shown). The envelope detector performs envelope detection of the signal output from the ultrasonic transmission/reception unit 101. The logarithmic converter relatively enhances a weak signal by logarithmically converting the amplitude of the detected signal. The analog/digital converter converts the output signal from this logarithmic converter into a digital signal to generate the first B-mode data. The first B-mode data is data which satisfies the input transmission/reception conditions and determined transmission/reception conditions. Assume that the first B-mode data generation unit 102 has a basic arrangement for generating B-mode data with a lower resolution than that generated by the second B-mode data generation unit 202. In this case, the term “resolution” refers to both a temporal resolution and a spatial resolution, and a resolution in the following description will mean both or one of them.

The first image generation unit 104 generates the first B-mode image based on the first B-mode data. The image generated by the first image generation unit 104 will be referred to as the first ultrasonic image hereinafter. In scan conversion, the first image generation unit 104 executes linear interpolation processing, frame correlation processing for a predetermined number of frames, and the like. The first image generation unit 104 has a basic arrangement for generating an image as compared with the second image generation unit 204.

The first image combination unit 106 combines the first ultrasonic image having undergone scan conversion with character information of various types of parameters, scale marks, and the like. The first image combination unit 106 outputs the combined first ultrasonic image to the first display unit 108. The first image combination unit 106 has a basic arrangement for combining images as compared with the second image combination unit 206.

The first display unit 108 displays the combined first ultrasonic image based on an output from the first image combination unit 106. Note that the first display unit 108 displays, for example, only the combined first ultrasonic image for the sake of reductions in power consumption and size.

The second unit 200 includes the second B-mode data generation unit 202, the Doppler data generation unit 201, the second image generation unit 204, the second image combination unit 206, the second display unit 208, a storage unit 203, a second control unit 209, a second interface unit 205, and the second input unit 207. Note that an external storage device 31 and a network may be connected to the second unit 200 via the second interface unit 205.

The second input unit 207 inputs transmission/reception conditions associated with the B mode and the Doppler mode, a selected imaging method, and the like.

The second control unit 209 includes a memory (not shown). The memory stores a plurality of reception delay patterns with different focus depths, control programs for the ultrasonic diagnostic apparatus 1 associated with the B mode and the Doppler mode, and the like. In response to the first output from the connection detection unit 119, the second control unit 209 controls the ultrasonic transmission/reception unit 101 of the first unit 10 and the second unit 200. At this time, in response to the first output, the first B-mode data generation unit 102, first image generation unit 104, and first image combination unit 106 of the first unit 10 are excluded from control targets. More specifically, the second control unit 209 reads out a reception delay pattern and a control program stored in the memory based on various kinds of instructions, commands, information, selections, and settings associated with the B mode and Doppler mode and Doppler mode which are input via the second input unit 207. The second control unit 209 controls the ultrasonic transmission/reception unit 101 and the second unit 200 in accordance with the input various kinds of instructions, commands, information, selections, and settings and the readout control program.

Note that when the first unit 10 is not connected to the second unit 200, the second control unit 209 allows to, for example, browse image data stored in the signal reading unit 13, the external storage device 31, and a server connected via a network and use a clinical application using stored image data in accordance with instructions issued by the operator via the second input unit 207.

The second B-mode data generation unit 202 acquires the reception signal generated by the ultrasonic transmission/reception unit 101 via the connection unit 117. The second B-mode data generation unit 202 generates the second B-mode data based on the acquired reception signal. The second B-mode data and the first B-mode data differ in the following manner. The first B-mode data is generated based on the reception signal generated while limitations are imposed on a plurality of transmission/reception conditions such as the number of transmission channels, the number of reception channels, the range of an ROI, a scanning range, a scanning line density, a frame rate, and a parallel reception function in accordance with the data processing ability of the first B-mode data generation unit 102. In contrast, the second B-mode data is generated based on the reception signal generated while no limitations are imposed on the plurality of transmission/reception conditions described above. Owing to this difference, the power consumption of the first B-mode data generation unit 102 is lower than that of the second B-mode data generation unit 202. In addition, the arrangement of the first B-mode data generation unit 102 is simpler than that of the second B-mode data generation unit 202, and allows downsizing. Furthermore, the second B-mode data generation unit 202 can generate the second B-mode data having a higher resolution than the first B-mode data.

The Doppler data generation unit 201 acquires the reception signal generated by the ultrasonic transmission/reception unit 101 via the connection unit 117. The connection unit 117 generates Doppler data based on the acquired reception signal.

The second image generation unit 204 generates an image based on at least one of the second B-mode data and the Doppler data. The image generated by the second image generation unit 204 will be referred to as the second ultrasonic image. The second image generation unit 204 executes arbitrary-order interpolation processing, enhancement and smoothing processing using various types of filters, frame correlation processing, and the like in scan conversion. Note that the second image generation unit 204 may include a memory which stores image data and execute reconstruction processing of a three-dimensional image and the like. The following is the difference between the processing performed by the first image generation unit 104 and the processing performed by the second image generation unit 204.

The first image generation unit 104 limits various types of processing for the first B-mode data. The second image generation unit 204 releases the control from the various types of processing described above. Owing to this difference, the power consumption of the first image generation unit 104 is lower than that of the second image generation unit 204. In addition, the arrangement of the first image generation unit 104 is simpler than that of the second image generation unit 204, and allows downsizing. Furthermore, the resolution of the image generated by the second image generation unit 204 is higher than that of the image generated by the first image generation unit 104.

The second image combination unit 206 combines the second ultrasonic image generated by the second image generation unit 204 with character information of various types of parameters, scale marks, and the like. The second image combination unit 206 generates a combined image by juxtaposing the second ultrasonic image and another ultrasonic image such as a past ultrasonic image of the object which is stored in the storage unit 203. The following is the difference between the first image combination unit 106 and the second image combination unit 206. The first image combination unit 106 combines the first ultrasonic image with information attached to the first ultrasonic image. On the other hand, the second image combination unit 206 generates the above combined image. Owing to this difference, the arrangement of the first image combination unit 106 is simpler than that of the second image combination unit 206, and allows downsizing.

The second display unit 208 displays the images combined by the second image combination unit 206. The second display unit 208 has a larger display area than the first display unit 108. In this case, a large display area means an increase in the number of display pixels. In general, with an increase in the number of display pixels, the size (area) of a display screen increases. Therefore, a large display area also means an increase in display screen size.

The following will describe the processing associated with the generation of ultrasonic images (to be referred to as ultrasonic image generation processing hereinafter) in the ultrasonic diagnostic apparatus 1 which can be separated into the first unit 10 having a basic arrangement for generating B-mode images having a predetermined resolution and the second unit 200 which generates the second ultrasonic image having a higher resolution than the first ultrasonic image.

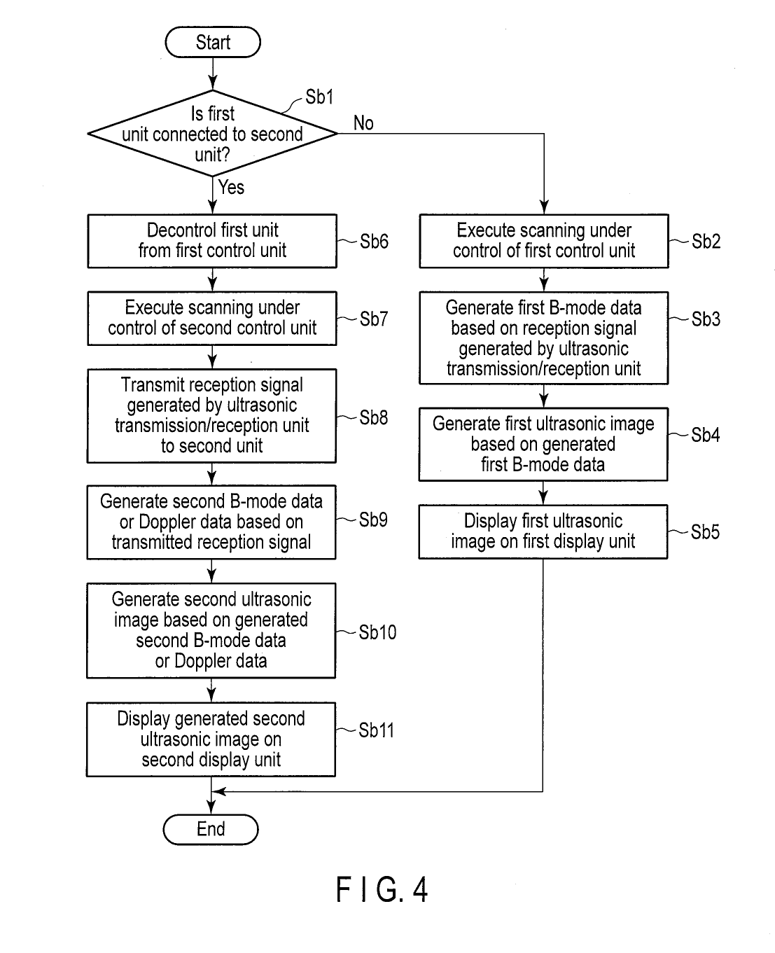

FIG. 4 is a flowchart showing an example of ultrasonic image generation processing.

If the first unit 10 is not connected to the second unit 200 via the connection unit 117 (step Sb1), the apparatus executes scanning under the control of the first control unit 113 (step Sb2). The apparatus generates the first B-mode data based on the reception signal generated by the ultrasonic transmission/reception unit 101 (step Sb3). The apparatus generates the first ultrasonic image based on the generated first B-mode data (step Sb4). The first display unit 108 displays the generated first ultrasonic image (step Sb5).

If the first unit 10 is connected to the second unit 200 via the connection unit 117 (step Sb1), the first control unit 113 decontrols the first unit 10 (step Sb6). The apparatus executes scanning under the control of the second control unit 209 (step Sb7). The reception signal generated by the ultrasonic transmission/reception unit 101 is transmitted to the second unit 200 via the connection unit 117 (step Sb8). The apparatus generates the second B-mode data or Doppler data based on the transmitted reception signal (step Sb9). The apparatus generates the second ultrasonic image based on the generated second B-mode data or Doppler data (step Sb10). The second display unit 208 displays the generated second ultrasonic image (step Sb11).

According to the above arrangement, the following effects can be obtained.

According to the ultrasonic diagnostic apparatus 1, it is possible to distribute the function associated with the generation of B-mode images and the function associated with display to the first unit 10 and the second unit 200. This can reduce the power consumption of the first unit 10 of the ultrasonic diagnostic apparatus 1, and improves the operating time of the first unit 10 of the ultrasonic diagnostic apparatus 1. In addition, this can simplify the arrangement of the first unit 10 of the ultrasonic diagnostic apparatus 1, leading to a reduction in the size of the first unit 10 of the ultrasonic diagnostic apparatus 1. Furthermore such reductions in power consumption and size can improve the operating time and portability. Therefore, this improves the convenience of the ultrasonic diagnostic apparatus 1 in home care, sports activities, disaster sites, and the like.

In addition, connecting the first unit 10 to the second unit 200 makes it possible to use the ultrasonic diagnostic apparatus 1 as a floor-standing type ultrasonic diagnostic apparatus, which is higher in function than a portable type ultrasonic diagnostic apparatus, in a hospital or the like. In addition, the ultrasonic diagnostic apparatus 1 can lead to a reduction in cost as compared with a case in which both a floor-standing type ultrasonic diagnostic apparatus and a portable type ultrasonic diagnostic apparatus are purchased. As described above, the ultrasonic diagnostic apparatus 1 can provide ultrasonic diagnostic services in accordance with conditions around objects and diagnostic purposes at low cost and with high efficiency.

### Third Embodiment

The third embodiment will be described below with reference to the accompanying drawing.

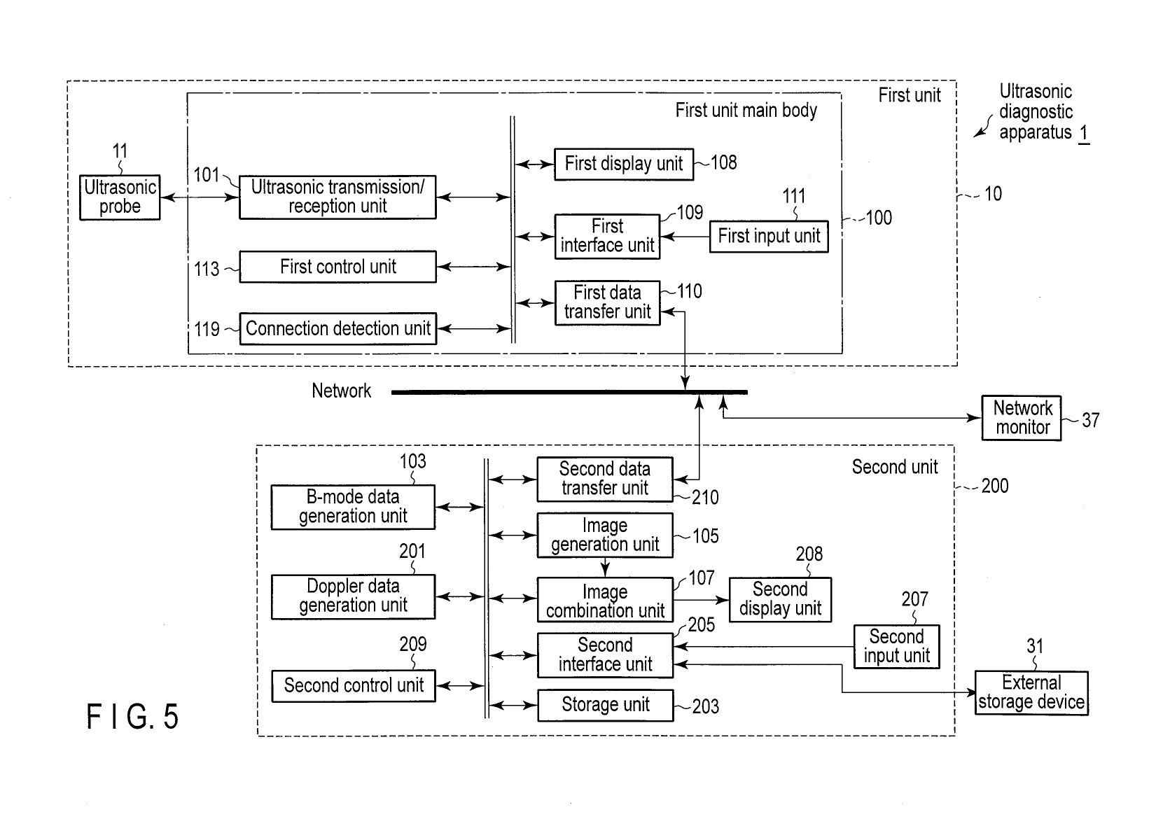

FIG. 5 is a block diagram showing the arrangement of an ultrasonic diagnostic apparatus according to the third embodiment.

This embodiment differs from the first and second embodiments in that it always connects a first unit 10 to a second unit 200 via a wired or wireless network, and cancels functional restrictions as in the first and second embodiments. Note that wired networks include, for example, a local area network (to be referred to as a LAN hereinafter) using an electric communication line and the Internet. Wireless networks include, for example, a wireless LAN and a satellite communication line. If, for example, a network is a hospital LAN, a hospital LAN terminal is installed in a medical ward or near the bed on which a patient is placed.

The first unit 10 includes an ultrasonic probe 11 and a first unit main body 100. The first unit main body 100 includes an ultrasonic transmission/reception unit 101, a first data transfer unit 110, a first input unit 111, a connection detection unit 119, a first control unit 113, a first interface unit 109, and a first input unit 111.

The first input unit 111 inputs various kinds of instructions, commands, information, selections, and settings associated with the B mode and the Doppler mode and transmission/reception conditions to the ultrasonic diagnostic apparatus 1.

The first control unit 113 includes a memory (not shown). The memory stores a plurality of reception delay patterns with different focus depths and control programs associated with ultrasonic transmission/reception. The first control unit 113 controls the ultrasonic transmission/reception unit 101 to transmit ultrasonic waves to an object in accordance with an instruction issued by the operator via the first input unit 111.

The ultrasonic transmission/reception unit 101 transmits ultrasonic waves to the object through the ultrasonic probe 11 under the control of the first control unit 113. The ultrasonic transmission/reception unit 101 receives reflected waves corresponding to the transmitted ultrasonic waves from the object. The ultrasonic transmission/reception unit 101 generates a reception signal based on the received reflected waves. The ultrasonic transmission/reception unit 101 outputs the generated reception signal to the first data transfer unit 110.

The first data transfer unit 110 transfers the reception signal output from the ultrasonic transmission/reception unit 101 to a second data transfer unit 210 of the second unit 200 via a network. The first data transfer unit 110 transfers, to a first display unit 108, the ultrasonic image transferred from the second data transfer unit 210 via the network. Note that if the band of the reception signal transferred from the first data transfer unit 110 exceeds a predetermined band, the first data transfer unit 110 can cut off the band by increasing the decimation rate of the reception signal. Note that the predetermined band ranges from, for example, several hundred Mbps to 1 Gbps. The decimation rate of a reception signal is, for example, the ratio of signals to be decimated to the reception signals generated by the ultrasonic transmission/reception unit 101. Alternatively, the ultrasonic transmission/reception unit 101 may transmit reception signals upon band limiting by compression processing.

The first display unit 108 displays the ultrasonic image transferred from the first data transfer unit 110.

The connection detection unit 119 detects the connection between the first unit 10 and the second unit 200 via a network. More specifically, first of all, the connection detection unit 119 performs, for example, pinging to detect the connection between the first data transfer unit 110 and the second data transfer unit 210. Subsequently, the connection detection unit 119 detects the connection between the first data transfer unit 110 and the second data transfer unit 210 in accordance with a response (reply) from the second data transfer unit 210.

The second unit 200 includes the second data transfer unit 210, a B-mode data generation unit 103, a Doppler data generation unit 201, an image generation unit 105, an image combination unit 107, a second display unit 208, a second control unit 209, a second interface unit 205, and a second input unit 207.

A second data transfer unit 210 transfers a reception signal from a first data transfer unit 110 via a network to the B-mode data generation unit 103 or the Doppler data generation unit 201. The second data transfer unit 210 transfers the image combined by the image combination unit 107 to the first data transfer unit 110 via the network.

The B-mode data generation unit 103 generates B-mode data based on the reception signal transferred from the second data transfer unit 210.

The Doppler data generation unit 201 generates Doppler data based on the reception signal transferred from the second data transfer unit 210.

The image generation unit 105 generates an ultrasonic image based on the B-mode data or Doppler data.

A storage unit 203 stores the B-mode data generated by the B-mode data generation unit 103, the Doppler data generated by the Doppler data generation unit 201, past ultrasonic image data associated with the object, and the like.

The image combination unit 107 combines the generated ultrasonic image with various types of parameter information, scale marks, and the like. Note that the image combination unit 107 may generate a combined image by juxtaposing the generated ultrasonic image and the past ultrasonic image of the object which is stored in the storage unit 203. The image combination unit 107 outputs the combined image data to the second data transfer unit 210.

The second display unit 208 displays the combined image generated by the image combination unit 107 in accordance with an instruction issued by the operator via the second input unit 207. Note that the second display unit 208 can also display an ultrasonic image or the like stored in an external storage device 31 connected via the second interface unit 205.

A network monitor 37 may be connected to the network which connects the first data transfer unit 110 to the second data transfer unit 210. The network monitor 37 displays information associated with the connection state between the first unit 10 and the second unit 200, the image combined by the image combination unit 107, the images stored in the storage unit 203 and external storage device 31, and the like. The operator can set display contents to be displayed on the network monitor 37 via the second input unit 207.

The following will describe processing associated with the generation of ultrasonic images (to be referred to as ultrasonic image generation processing hereinafter) in the ultrasonic diagnostic apparatus 1 including the first unit 10 and the second unit 200 which are connected via the network.

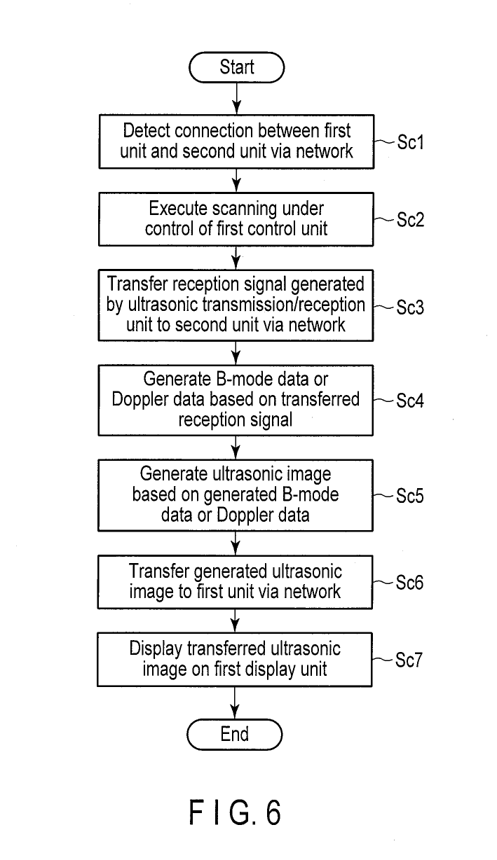

FIG. 6 is a flowchart showing an example of a procedure for ultrasonic image generation processing according to the third embodiment.

When the connection detection unit 119 detects the connection between the first unit 10 and the second unit 200 (step Sc1), the apparatus executes scanning under the control of the first control unit 113 (step Sc2). The reception signal generated by the ultrasonic transmission/reception unit 101 is transferred to the B-mode data generation unit 103 of the second unit 200 or the Doppler data generation unit 201 via the network (step Sc3). The apparatus generates B-mode data or Doppler data based on the transferred reception signal (step Sc4). The apparatus generates an ultrasonic image based on the generated B-mode data or Doppler data (step Sc5). The generated ultrasonic image is transferred to the first unit 10 via the network (step Sc6). The first display unit 108 displays the transferred ultrasonic image (step Sc7).

According to the above arrangement, the following effects can be obtained.

According to the ultrasonic diagnostic apparatus 1, it is possible to decrease the number of constituent elements mounted in the first unit 10 as compared with the prior art by connecting the first unit 10, which generates reception signals, to the second unit 200, which generates ultrasonic images based on transferred reception signals, via a network. This can reduce the power consumption of the first unit 10. Reducing the power consumption of the first unit 10 improves the operating time of the first unit 10. Reducing the number of constituent elements mounted in the first unit 10 contributes to the downsizing of the first unit 10. In addition, such reductions in power consumption and size can improve the operating time and portability. Therefore, this improves the convenience of the ultrasonic diagnostic apparatus 1 in home care, sports activities, disaster sites, and the like.

Since the first unit 10 is connected to the second unit 200 via the network, the ultrasonic diagnostic apparatus 1 can be used as a floor-standing ultrasonic diagnostic apparatus higher in function than a portable type ultrasonic diagnostic apparatus. If, for example, the network is a satellite communication line, it is possible to use the ultrasonic diagnostic apparatus 1 as an ultrasonic diagnostic apparatus having high functionality like a floor-standing ultrasonic diagnostic apparatus in all places.

In addition, the ultrasonic diagnostic apparatus 1 can lead to a reduction in cost as compared with a case in which both a floor-standing type ultrasonic diagnostic apparatus and a portable type ultrasonic diagnostic apparatus are purchased. As described above, the ultrasonic diagnostic apparatus 1 can provide ultrasonic diagnostic services in accordance with conditions around objects and diagnostic purposes at low cost and with high efficiency.

Furthermore, this embodiment can provide an ultrasonic diagnostic apparatus as a thin client system.

### Fourth Embodiment

The fourth embodiment will be described below with reference to the accompanying drawing.

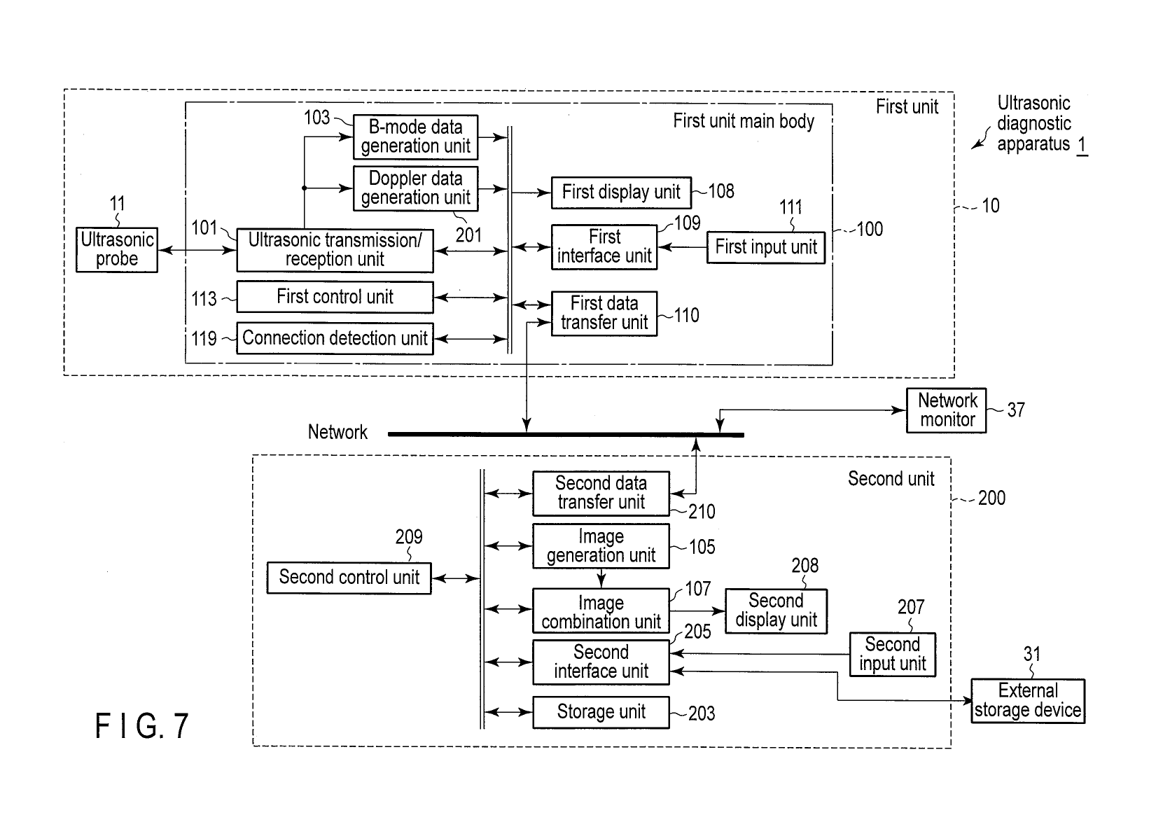

FIG. 7 is a block diagram showing the arrangement of an ultrasonic diagnostic apparatus according to the fourth embodiment.

The fourth embodiment differs from the third embodiment in that a first unit 10 is equipped with a B-mode data generation unit 103 and a Doppler data generation unit 201. The B-mode data generation unit 103 and an image generation unit 105 respectively generate B-mode data and Doppler data based on the reception signals generated by an ultrasonic transmission/reception unit 101. The B-mode data and the Doppler data are sufficiently smaller in band than output signals from the ultrasonic transmission/reception unit 101. This makes it possible to transfer the B-mode data and the Doppler data in a predetermined band (e.g., several hundred Mbps to 1 Gbps). Alternatively, this eliminates the necessity to reduce the amount of reception signals as much as in the third embodiment. Alternatively, since there is no need to highly compress signals, it is possible to perform band compressing by a simple compression technique with less CPU load. Alternatively, it is possible to greatly reduce the transfer load by further reducing the signal band by high compression.

The first unit 10 includes an ultrasonic probe 11 and a first unit main body 100. The first unit main body 100 includes the ultrasonic transmission/reception unit 101, the B-mode data generation unit 103, the Doppler data generation unit 201, a first data transfer unit 110, a first display unit 108, a connection detection unit 119, a first control unit 113, a first interface unit 109, and a first input unit 111.

The first input unit 111 inputs, to an ultrasonic diagnostic apparatus 1, various kinds of instructions, commands, information, selections, and settings associated with the B mode and Doppler mode, ultrasonic transmission/reception conditions, and the like.

The first control unit 113 includes a memory (not shown). The memory stores a plurality of reception delay patterns with different focus depths, control programs associated with the ultrasonic transmission/reception, and the like. The first control unit 113 controls the ultrasonic transmission/reception unit 101 to transmit ultrasonic waves to an object in accordance with an instruction issued by the operator via the first input unit 111.

The ultrasonic transmission/reception unit 101 transmits ultrasonic waves to the object through the ultrasonic probe 11 under the control of the first control unit 113. The ultrasonic transmission/reception unit 101 receives reflected waves corresponding to the transmitted ultrasonic waves from the object. The ultrasonic transmission/reception unit 101 generates a reception signal based on the received reflected waves. The ultrasonic transmission/reception unit 101 outputs the generated reception signal to the B-mode data generation unit 103 and the Doppler data generation unit 201.

The B-mode data generation unit 103 generates B-mode data based on the reception signal output from the ultrasonic transmission/reception unit 101.

The Doppler data generation unit 201 generates Doppler data based on the reception signal output from the ultrasonic transmission/reception unit 101.

The first data transfer unit 110 transfers B-mode data and Doppler data to a second data transfer unit 210 of a second unit 200 via a network. A first data transfer unit 110 transfers, to the first display unit 108, the ultrasonic image transferred from the second data transfer unit 210 (to be described later) via the network. Note that the bands of the B-mode data and Doppler data transferred from the first data transfer unit 110 range from, for example, 100 Mbps to 300 Mbps. This alleviates data transmission conditions as compared with the third embodiment.

The first display unit 108 displays the ultrasonic image transferred from the first data transfer unit 110.

The connection detection unit 119 detects the connection between the first unit 10 and the second unit 200 via a network.

The second unit 200 includes the second data transfer unit 210, the image generation unit 105, an image combination unit 107, a second display unit 208, a second control unit 209, a second interface unit 205, and a second input unit 207.

The second data transfer unit 210 transfers, to the image generation unit 105, the B-mode data and Doppler data transferred from the first data transfer unit 110 via the network. The second data transfer unit 210 transfers the image combined by the image combination unit 107 (to be described later) to the first data transfer unit 110 via the network.

The image generation unit 105 generates an ultrasonic image based on the transferred B-mode data or Doppler data.

A storage unit 203 stores transferred B-mode data and Doppler data, past ultrasonic image data associated with objects, and the like.

The image combination unit 107 combines a generated ultrasonic image with various kinds of parameter information, scale marks, and the like. Note that the image combination unit 107 may generate a combined image by juxtaposing the generated ultrasonic image and the past ultrasonic image of the object which is stored in a storage unit 13. The image combination unit 107 outputs the combined image data to the second data transfer unit 210.

The second display unit 208 displays the combined image generated by the image combination unit 107 in accordance with an instruction issued by the operator via the second input unit 207. Note that the second display unit 208 can also display an ultrasonic image or the like stored in an external storage device 31 connected via the second interface unit 205.

A network monitor 37 may be connected to the network which connects the first data transfer unit 110 to the second data transfer unit 210. The network monitor 37 displays information associated with the connection state between the first unit 10 and the second unit 200, the image combined by the image combination unit 107, the images stored in the storage unit 13 and an external storage device 31, and the like. The operator can set display contents to be displayed on the network monitor 37 via the second input unit 207.

The following will describe processing associated with the generation of ultrasonic images (to be referred to as ultrasonic image generation processing hereinafter) in the ultrasonic diagnostic apparatus 1 including the first unit 10 and the second unit 200 which are connected via the network.

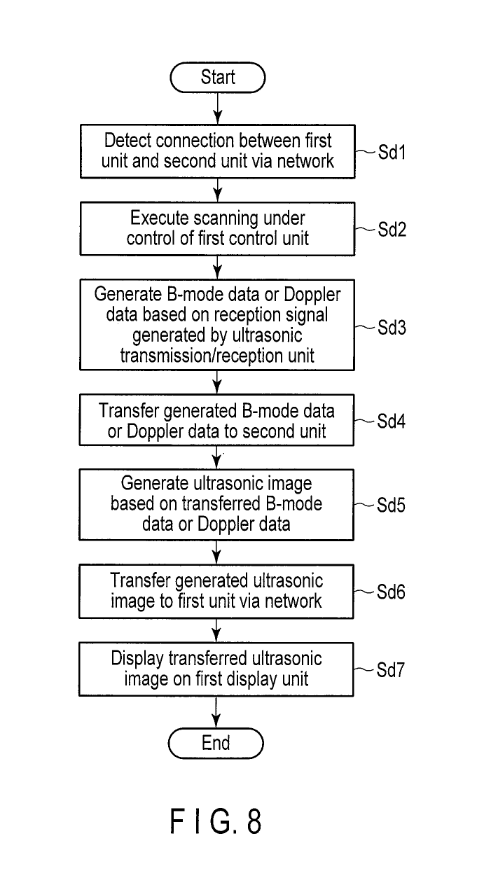

FIG. 8 is a flowchart showing an example of a procedure for ultrasonic image generation processing according to the fourth embodiment.

When the connection detection unit 119 detects the connection between the first unit 10 and the second unit 200 (step Sd1), the apparatus executes scanning under the control of the first control unit 113 (step Sd2). The apparatus generates B-mode data or Doppler data based on the reception signal generated by the ultrasonic transmission/reception unit 101 (step Sd3). The generated B-mode data or Doppler is transferred to the second unit 200 via the network (step Sd4). The apparatus generates an ultrasonic image based on the transferred B-mode data or Doppler data (step Sd5). The generated ultrasonic image data is transferred to the first unit 10 via the network (step Sd6). The first display unit 108 displays an ultrasonic image based on the transferred ultrasonic image data (step Sd7).

According to the above arrangement, the following effects can be obtained.

According to the ultrasonic diagnostic apparatus 1, it is possible to reduce the amount of data transferred from the first unit 10 to the second unit 200 by connecting the first unit 10, which generates B-mode data or Doppler based on reception signals, to the second unit 200, which generates ultrasonic images based on transferred B-mode data or Doppler data, via the network. This can generate good ultrasonic images without cutting off generated reception signals.

Since the first unit 10 is connected to the second unit 200 via the network, the ultrasonic diagnostic apparatus 1 can also be used as a floor-standing ultrasonic diagnostic apparatus higher in function than a portable type ultrasonic diagnostic apparatus. If, for example, the network is a satellite communication line, it is possible to use the ultrasonic diagnostic apparatus 1 as an ultrasonic diagnostic apparatus having high functionality like a floor-standing ultrasonic diagnostic apparatus in all places.

In addition, the ultrasonic diagnostic apparatus 1 can lead to a reduction in cost as compared with a case in which both a floor-standing type ultrasonic diagnostic apparatus and a portable type ultrasonic diagnostic apparatus are purchased. As described above, the ultrasonic diagnostic apparatus 1 can provide ultrasonic diagnostic services in accordance with conditions around objects and diagnostic purposes at low cost and with high efficiency.

Furthermore, this embodiment can provide an ultrasonic diagnostic apparatus as a thin client system.

### Fifth Embodiment

The fifth embodiment will be described below with reference to the accompanying drawing.

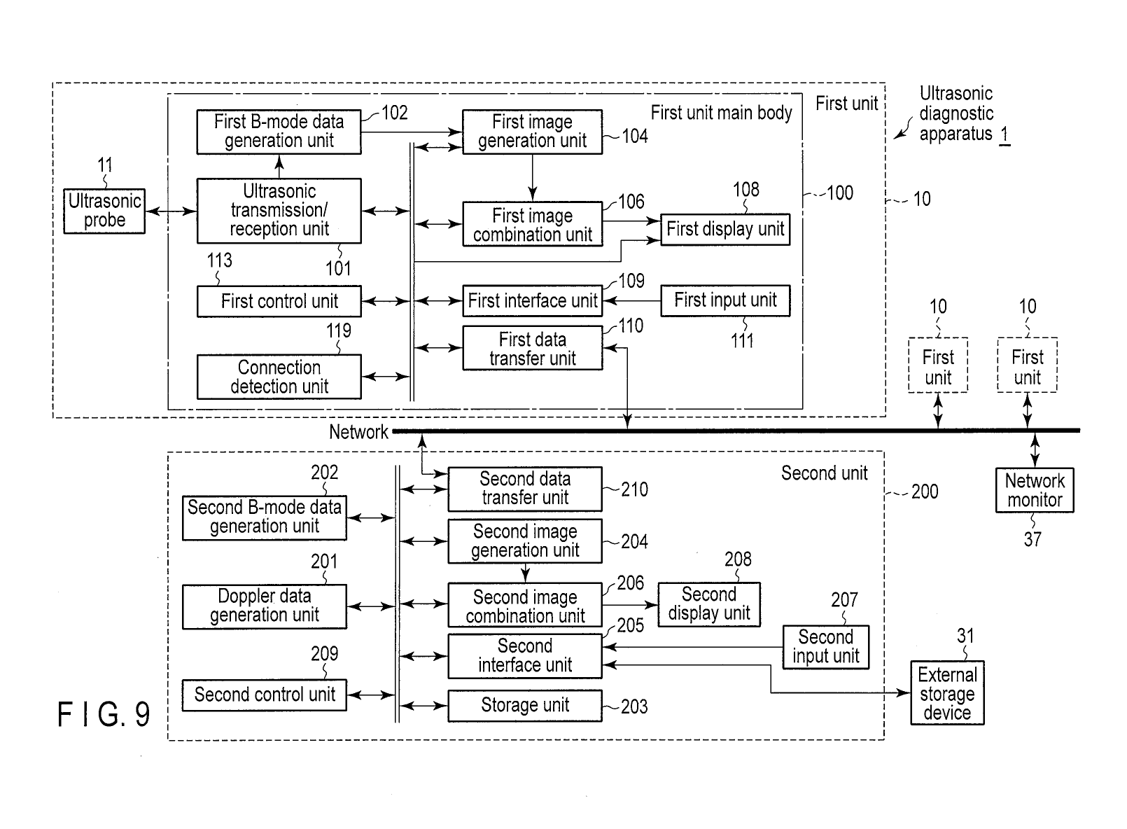

FIG. 9 is a block diagram showing the arrangement of an ultrasonic diagnostic apparatus according to the fifth embodiment.

The fifth embodiment differs from the second embodiment in that the first and second units in the second embodiment are connected via a network. Note that a plurality of first units can be connected to the network.

A first unit 10 includes an ultrasonic probe 11 and a first unit main body 100. The first unit main body 100 includes an ultrasonic transmission/reception unit 101, a first B-mode data generation unit 102, a first image generation unit 104, a first image combination unit 106, a first display unit 108, a first data transfer unit 110, a connection detection unit 119, a first control unit 113, a first interface unit 109, and first input unit 111.

The first input unit 111 is connected to the first interface unit 109. The first input unit 111 inputs, to the ultrasonic diagnostic apparatus 1, a plurality of transmission/reception conditions such as the number of transmission channels associated with the B mode, the number of reception channels, the range of an ROI, a scanning range, a scanning line density, a frame rate, and the number of scanning lines associated with a parallel reception function.

The first control unit 113 includes a memory (not shown). The memory of the first control unit 113 stores a plurality of reception delay patterns with different focus depths, control programs for the first unit main body 100 which are associated with the B mode, and the like. Upon receiving at least one of a plurality of transmission/reception conditions such as the number of transmission channels, the number of reception channels, the range of an ROI, a scanning range, a scanning line density, a frame rate, and the number of scanning lines associated with a parallel simultaneous reception function, the first control unit 113 determines other transmission/reception conditions based on the data processing ability of the first B-mode data generation unit 102 (to be described later). The first control unit 113 reads out a reception delay pattern and control program stored in the memory based on the determined transmission/reception conditions. The first control unit 113 controls the first unit 10 of the ultrasonic diagnostic apparatus 1 in accordance with the input and determined transmission/reception conditions and the readout control program.

Note that the first control unit 113 may read out a control program from the memory based on input transmission/reception conditions. At this time, the first control unit 113 may decimate reception signals output from an ultrasonic transmission/reception unit 101 so as to match the determined transmission/reception conditions.

When the connection detection unit 119 (to be described later) detects the connection between the first unit 10 and second unit 200 via the network, the first control unit 113 releases the control from the first B-mode data generation unit 102, first image generation unit 104, and first image combination unit 106 mounted in the first unit 10. More specifically, the first control unit 113 releases the control from the first B-mode data generation unit 102, the first image generation unit 104, and the first image combination unit 106 based on the first output from the connection detection unit 119. The first output is information indicating that the first unit 10 is connected to the second unit 200 via the network.

When the connection detection unit 119 (to be described later) detects the disconnection between the first unit 10 and the second unit 200, the first control unit 113 starts controlling the respective units included in the first unit 10. More specifically, the first control unit 113 starts controlling the respective units included in the first unit 10 based on the second output from the connection detection unit 119 (to be described later). The second output is information associated with the disconnection between the first unit 10 and the second unit 200.

The connection detection unit 119 detects the connection or disconnection between the first unit 10 and the second unit 200 via the network. More specifically, the connection detection unit 119 performs, for example, pinging to detect the connection between the first data transfer unit 110 (to be described later) and a second data transfer unit 210. Subsequently, the connection detection unit 119 detects the connection between the first data transfer unit 110 and the second data transfer unit 210 in accordance with a response (reply) from the second data transfer unit 210. Upon detecting the connection between the first unit 10 and the second unit 200, the connection detection unit 119 outputs information (first output) associated with connection to the first control unit 113 and the second control unit 209. When the first unit 10 is not connected to the second unit 200, the connection detection unit 119 outputs information (second output) associated with the disconnection to the first control unit 113.

The ultrasonic transmission/reception unit 101 transmits ultrasonic waves to the object through the ultrasonic probe 11. The ultrasonic transmission/reception unit 101 receives reflected waves corresponding to the transmitted ultrasonic waves from the object. The ultrasonic transmission/reception unit 101 generates a reception signal based on the received reflected waves. The ultrasonic transmission/reception unit 101 outputs the generated reception signal to the first B-mode data generation unit 102 under the control of the first control unit 113. The ultrasonic transmission/reception unit 101 outputs the generated reception signal to the first data transfer unit 110 (to be described later) under the control of a second control unit 209 (to be described later).

While the first unit 10 is connected to the second unit 200 via the network, the first data transfer unit 110 transfers the reception signal output from the ultrasonic transmission/reception unit 101 to the second data transfer unit 210 via the network. The first data transfer unit 110 transfers, to the first display unit 108, the ultrasonic image transferred from the second data transfer unit 210 via the network. Note that when the band of the reception signal transferred from the first data transfer unit 110 exceeds a predetermined band, the first data transfer unit 110 can cut off the band by increasing the decimation rate of reception signals. Note that the predetermined band ranges from, for example, several hundred Mbps to 1 Gbps. The decimation rate of reception signals is, for example, the ratio of signals to be decimated to the reception signals generated by the ultrasonic transmission/reception unit 101. Alternatively, the first data transfer unit 110 may transmit reception signals upon band limiting by compression processing.

The first B-mode data generation unit 102 generates the first B-mode data based on the reception signal output from the ultrasonic transmission/reception unit 101. The first B-mode data generation unit 102 generates the first B-mode data to the first image generation unit 104.

The first image generation unit 104 generates the first B-mode image based on the first B-mode data output from the first B-mode data generation unit 102. The image generated by the first image generation unit 104 will be referred to as the first ultrasonic image. In scan conversion, the first image generation unit 104 executes linear interpolation processing, frame correlation processing for a predetermined number of frames, and the like. The first image generation unit 104 has a basic arrangement for generating an image as compared with the second image generation unit 204.

The first image combination unit 106 combines the first ultrasonic image having undergone scan conversion with character information of various types of parameters, scale marks, and the like. The first image combination unit 106 outputs the combined first ultrasonic image to the first display unit 108. The first image combination unit 106 has a basic arrangement for combining images as compared with the second image combination unit 206.

The first display unit 108 displays the combined first ultrasonic image based on an output from the first image combination unit 106. Note that the first display unit 108 displays, for example, only the combined first ultrasonic image to reduce the power consumption and the size.

The second unit 200 includes a Doppler data generation unit 201, a second B-mode data generation unit 202, a storage unit 203, a second image generation unit 204, a second interface unit 205, a second image combination unit 206, a second input unit 207, a second display unit 208, a second control unit 209, and a second data transfer unit 210.

The second input unit 207 inputs transmission/reception conditions associated with the B mode and the Doppler, a selected imaging method, and the like.

The second control unit 209 includes a memory (not shown). The memory stores a plurality of reception delay patterns with different focus depths, control programs for the ultrasonic diagnostic apparatus 1 associated with the B mode and the Doppler mode, and the like. In response to the first output from the connection detection unit 119, the second control unit 209 controls the ultrasonic transmission/reception unit 101 of the first unit 10 and the second unit 200. At this time, the first B-mode data generation unit 102, first image generation unit 104, and first image combination unit 106 of the first unit 10 are excluded from control targets in response to the first output. More specifically, the second control unit 209 reads out a reception delay pattern and a control program stored in the memory based on various kinds of instructions, commands, information, selections, and settings associated with the B mode and Doppler mode which are input via the second input unit 207.

Note that when the first unit 10 is not connected to the second unit 200, the second control unit 209 allows to, for example, browse image data stored in the storage unit 203, the external storage device 31, and a server connected via a network and use a clinical application using stored image data in accordance with instructions issued by the operator via the second input unit 207.

The second data transfer unit 210 transfers a reception signal from the first data transfer unit 110 via the network to the second B-mode data generation unit 202 or Doppler data generation unit 201 (to be described later). The second data transfer unit 210 transfers the image data combined by the second image combination unit 206 (to be described later) to the first data transfer unit 110 via the network.

The second B-mode data generation unit 202 acquires via the second data transfer unit 210 the reception signal generated by the ultrasonic transmission/reception unit 101. The second B-mode data generation unit 202 generates the second B-mode data based on the acquired reception signal. The following is the difference between the second B-mode data and the first B-mode data. The first B-mode data is generated based on the reception signal generated while limitations are imposed on a plurality of transmission/reception conditions such as the number of transmission channels, the number of reception channels, the range of an ROI, a scanning range, a scanning line density, a frame rate, and a parallel reception function in accordance with the data processing ability of the first B-mode data generation unit 102. In contrast, the second B-mode data is generated based on the reception signal generated while no limitations are imposed on the plurality of transmission/reception conditions described above. Owing to this difference, the power consumption of the first B-mode data generation unit 102 is lower than that of the second B-mode data generation unit 202. In addition, the arrangement of the first B-mode data generation unit 102 is simpler than that of the second B-mode data generation unit 202, and allows downsizing. Furthermore, the second B-mode data generation unit 202 can generate the second B-mode data having a higher resolution than the first B-mode data.

The Doppler data generation unit 201 acquires via the second data transfer unit 210 the reception signal generated by the ultrasonic transmission/reception unit 101. The Doppler data generation unit 201 generates Doppler data based on the acquired reception signal.

The second image generation unit 204 generates an image based on at least one of the second B-mode data and the Doppler data. The image generated by the second image generation unit 204 will be referred to as the second ultrasonic image. The second image generation unit 204 executes arbitrary-order interpolation processing, enhancement and smoothing processing using various types of filters, frame correlation processing, and the like in scan conversion. Note that the second image generation unit 204 may include a memory which stores image data and execute reconstruction processing of a three-dimensional image and the like. The following is the difference between the processing performed by the first image generation unit 104 and the processing performed by the second image generation unit 204.

The first image generation unit 104 limits various types of processing for the first B-mode data. The second image generation unit 204 releases the control from the various types of processing described above. Owing to this difference, the power consumption of the first image generation unit 104 is lower than that of the second image generation unit 204. In addition, the arrangement of the first image generation unit 104 is simpler than that of the second image generation unit 204, and allows downsizing. Furthermore, the resolution of the image generated by the second image generation unit 204 is higher than that of the image generated by the first image generation unit 104.

The second image combination unit 206 combines the second ultrasonic image generated by the second image generation unit 204 with character information of various types of parameters, scale marks, and the like. Note that the second image combination unit 206 may generate a combined image by juxtaposing the second ultrasonic image and another ultrasonic image such as a past ultrasonic image of the object which is stored in the storage unit 203. The following is the difference between the first image combination unit 106 and the second image combination unit 206. The first image combination unit 106 combines the first ultrasonic image with information attached to the first ultrasonic image. On the other hand, the second image combination unit 206 generates the above combined image. Owing to this difference, the arrangement of the first image combination unit 106 is simpler than that of the second image combination unit 206, and allows downsizing.

The second display unit 208 displays the images combined by the second image combination unit 206. The second display unit 208 has a larger display area than the first display unit 108.

The above description has referred to the real-time processing to be performed when the first unit 10 is connected to the second unit 200 via the network. However, a storage unit (not shown) in the first unit 10 may temporarily store an output signal from the ultrasonic transmission/reception unit 101 or the first image generation unit 104 before the first unit 10 is connected to the second unit 200. When the connection is confirmed, the data temporarily stored in the first unit 10 may be transferred to improve the image quality by complicated filter processing by the processing unit in the second unit 200. Alternatively, the apparatus may execute applications such as data analysis and application measurement based on output signals.

The following will describe the processing associated with the generation of ultrasonic images (to be referred to as ultrasonic image generation processing hereinafter) in the ultrasonic diagnostic apparatus 1 which can be separated into the first unit 10, which generates B-mode images having a predetermined resolution, and the second unit 200, which generates the second ultrasonic image having a higher resolution than the first ultrasonic image, via a network.

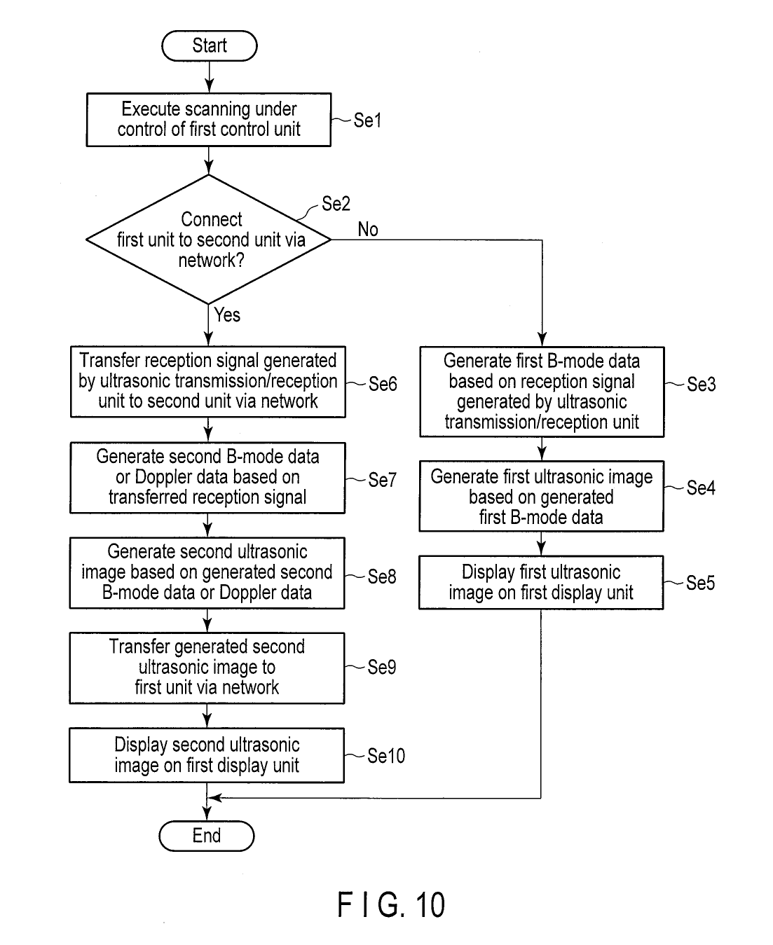

FIG. 10 is a flowchart showing an example of a procedure for ultrasonic image generation processing.

The apparatus executes scanning under the control of the first control unit 113 (step Se1). If the first unit 10 is not connected to the second unit 200 via a network at this time (step Se2), the apparatus executes processing in step Se3, processing in step Se4, and processing in step Se5.

If the first unit 10 is connected to the second unit 200 via the network (step Se2), the reception signal generated by the ultrasonic transmission/reception unit 101 is transferred to the second unit 200 via the network (step Se6). The apparatus generates the second B-mode data or Doppler data based on the transferred reception signal (step Se7). The apparatus generates the second ultrasonic image based on the generated second B-mode data or Doppler data (step Se8). The generated second ultrasonic image is transferred to the first unit 10 via the network (step Se9). The first display unit 108 displays the transferred second ultrasonic image (step Se10).

According to the above arrangement, the following effects can be obtained.

According to the ultrasonic diagnostic apparatus 1, it is possible to distribute the respective units associated with the generation and display of B-mode images to the first unit 10 and the second unit 200. This can reduce the power consumption of the first unit 10 of the ultrasonic diagnostic apparatus 1. This can improve the operating time. This can simplify the arrangement of the first unit 10, leading to a reduction in the size of the first unit 10. Furthermore such reductions in power consumption and size can improve the operating time and portability. Therefore, this improves the convenience of the ultrasonic diagnostic apparatus 1 in home care, sports activities, disaster sites, and the like.

Since the first unit 10 is connected to the second unit 200 via the network, the ultrasonic diagnostic apparatus 1 can also be used as a floor-standing ultrasonic diagnostic apparatus higher in function than a portable type ultrasonic diagnostic apparatus. If, for example, the network is a satellite communication line, it is possible to use the ultrasonic diagnostic apparatus 1 as an ultrasonic diagnostic apparatus having high functionality like a floor-standing ultrasonic diagnostic apparatus in all places.

In addition, the ultrasonic diagnostic apparatus 1 can lead to a reduction in cost as compared with a case in which both a floor-standing type ultrasonic diagnostic apparatus and a portable type ultrasonic diagnostic apparatus are purchased. As described above, the ultrasonic diagnostic apparatus 1 can provide ultrasonic diagnostic services in accordance with conditions around objects and diagnostic purposes at low cost and with high efficiency.

According to this embodiment, a plurality of the first units 10 can be connected to the second unit 200 within a limit allowed by the performance of the second unit 200. Providing an ultrasonic diagnostic apparatus as a thin client system can further improve the cost benefit.

### Sixth Embodiment

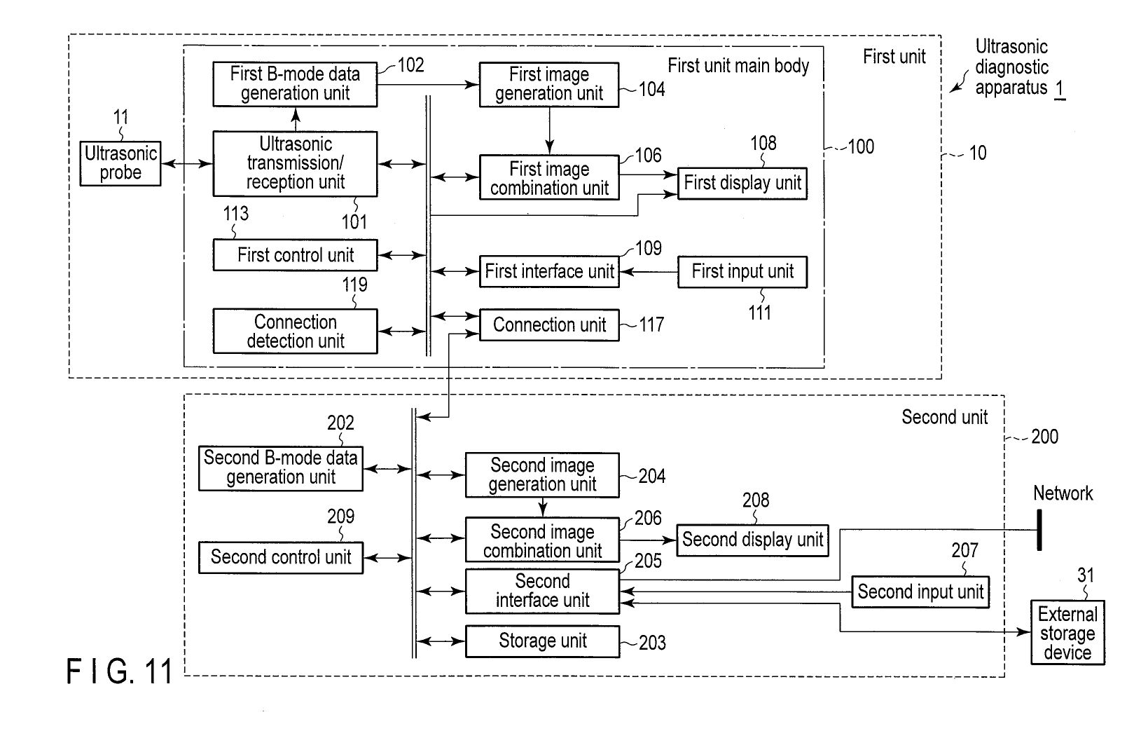

FIG. 11 is a block diagram showing the arrangement of an ultrasonic diagnostic apparatus 1 according to the sixth embodiment.

A first unit 10 is equipped with a CPU capable of implementing image quality, image mode, and performance allowable as a portable type ultrasonic diagnostic apparatus, a compact battery, and the like.

A second unit 200 is equipped with a high-output power supply, a function/performance expansion board, a mass-storage device, a large-capacity battery, and the like (none of which are shown).

A first B-mode data generation unit 102 generates the first B-mode data based on a reception signal. The first B-mode data generation unit 102 has a processing function and operation speed which are minimum necessary for the generation of the first B-mode data. For example, the first B-mode data generation unit 102 has a detection processing function of a full-wave rectification scheme with a light load in terms of hardware.

A first image generation unit 104 generates the first B-mode image based on the first B-mode data. The first image generation unit 104 has a processing function and operation speed which are minimum necessary for the generation of the first B-mode data. The generated first B-mode image has image quality (resolution), field angle, and the like which are minimum necessary for an ultrasonic image.

A first control unit 113 controls an ultrasonic transmission/reception unit 101 to obtain a reception signal associated with the generation of the first B-mode data. More specifically, the first control unit 113 controls the ultrasonic transmission/reception unit 101 to apply a driving voltage to each of a plurality of piezoelectric transducer located near the center of the opening of an ultrasonic probe 11. That is, the ultrasonic transmission/reception unit 101 applies a driving voltage to each of some of the piezoelectric transducers of the ultrasonic probe 11 under the control of the first control unit 113. The scanning range is limited under the control of the first control unit 113.

When the second unit 200 is connected to the first unit 10 via a connection unit 117, the first control unit 113 releases the control from each unit mounted in a first unit main body 100. That is, the first control unit 113 releases the control right of each unit except for the first B-mode data generation unit 102 and first image generation unit 104 (to be referred to as the first ultrasonic image generation unit hereinafter). When the first unit 10 is connected to the second unit 200, the control right may be moved to a second control unit 209.

A storage unit 203 stores the transferred medical image and volume data generated by other medical image diagnostic apparatuses.

The second control unit 209 includes a digital signal processor (to be referred to as a DSP hereinafter) having a higher operation speed than the DSP mounted in the first control unit 113. When the second unit 200 is connected to the first unit 10 via the connection unit 117, the second control unit 209 controls the ultrasonic transmission/reception unit 101 as well as controlling the respective units of the second unit 200. More specifically, the second control unit 209 controls the ultrasonic transmission/reception unit 101 to apply driving voltages to piezoelectric transducers larger in number than those to which driving voltages are applied in the first unit 10 alone. This increases the number of channels driven and improves the S/N ratio. The second control unit 209 executes parallel simultaneous reception by controlling the ultrasonic transmission/reception unit 101. In addition, the performance of the digital beam former implemented under the control of the second control unit 209 becomes higher than that of the digital beam former implemented under the control of first control unit 113.

Note that the second control unit 209 may control a second image generation unit 204 to execute various types of real-time clinical application processing. Clinical applications include, for example, an application for medical treatment support by positioning the medical images generated by other medical image diagnostic apparatuses with the generated ultrasonic images. If, for example, an elastic image contains the heart of an object, the second control unit 209 may have a function associated with cardiac function analysis. The function associated with cardiac function analysis is, for example, a function associated with a two-dimensional or three-dimensional myocardial strain. Note that when the first unit 10 is disconnected from the second unit 200, the control right of the first ultrasonic image generation unit moves from the second control unit 209 to the first control unit 113.

A second B-mode data generation unit 202 generates the second B-mode data larger in data amount than the first B-mode data based on a reception signal. The operation speed of the second B-mode data generation unit 202 is higher than that of the first B-mode data generation unit 102. This improves the throughput of an echo processor per unit time.

The second image generation unit 204 generates the second B-mode image having a higher resolution than the first B-mode image based on the second B-mode data. Note that the second image generation unit 204 generates a rendering image based on the second B-mode data. At this time, the second image generation unit 204 has a rendering function. The second image generation unit 204 is equipped with a graphics processing unit (to be referred to as a GPU hereinafter) higher in operation speed than the GPU mounted in the first image generation unit 104. The second image generation unit 204 has a function of executing real-time image quality improving processing. The real-time image quality improving is, for example, two-dimensional or three-dimensional nonlinear filter processing. This nonlinear filter processing can reduce noise while leaving edges in an image. Note that the second image generation unit 204 may perform four-dimensional processing, quantification processing, and the like.

Note that the second image generation unit 204 can generate an elastic image (elastography) based on the second B-mode data.

The second image generation unit 204 generates a tomographic image of almost the same slice as that of the second B-mode image by using an output from a position sensor (magnetic sensor or photosensor) (not shown) provided for the ultrasonic probe 11 based on the volume data stored in the storage unit 203. The tomographic image is output to a second image combination unit 206.

The second B-mode data generation unit and the second image generation unit will be collectively referred to as the second ultrasonic image generation unit hereinafter.

The second image combination unit 206 displays the tomographic image and the second B-mode image side by side. At this time, the update rate of tomographic images to be displayed differs from that of second B-mode images.

According to the above arrangement, the following effects can be obtained.

The ultrasonic diagnostic apparatus 1 is configured to be separated into the first unit 10 having basic performance and function associated with the generation of B-mode images and the second unit 200 which has higher function and performance than the basic function and performance and generate B-mode images. With this arrangement, when the first unit 10 is connected to the second unit 200, the second ultrasonic image generation unit generates the second B-mode image instead of the first ultrasonic image generation unit. This improves the performance and function as compared with when the first unit 10 is used alone. In addition, this embodiment allows the expansion of a clinical application. As described above, according to this embodiment, connecting the first unit 10 to the second unit 200 will improve the throughput and expand the function.

In addition, it is possible to reduce the power consumption of the first unit 10 of the ultrasonic diagnostic apparatus 1. This improves the operating time of the first unit 10 of the ultrasonic diagnostic apparatus 1. In addition, this can simplify the arrangement of the first unit 10 of the ultrasonic diagnostic apparatus 1, leading to a reduction in the size of the first unit 10 of the ultrasonic diagnostic apparatus 1. Furthermore such reductions in power consumption and size can improve the operating time and portability. Therefore, this improves the convenience of the ultrasonic diagnostic apparatus 1 in home care, sports activities, disaster sites, and the like.

In addition, connecting the first unit 10 to the second unit 200 makes it possible to use the ultrasonic diagnostic apparatus 1 as a high-function floor-standing type ultrasonic diagnostic apparatus in a hospital or the like. In addition, the ultrasonic diagnostic apparatus 1 can lead to a reduction in cost as compared with a case in which both a floor-standing type ultrasonic diagnostic apparatus and a portable type ultrasonic diagnostic apparatus are purchased. As described above, the ultrasonic diagnostic apparatus 1 can provide ultrasonic diagnostic services in accordance with conditions around objects and diagnostic purposes at low cost and with high efficiency.

### Seventh Embodiment

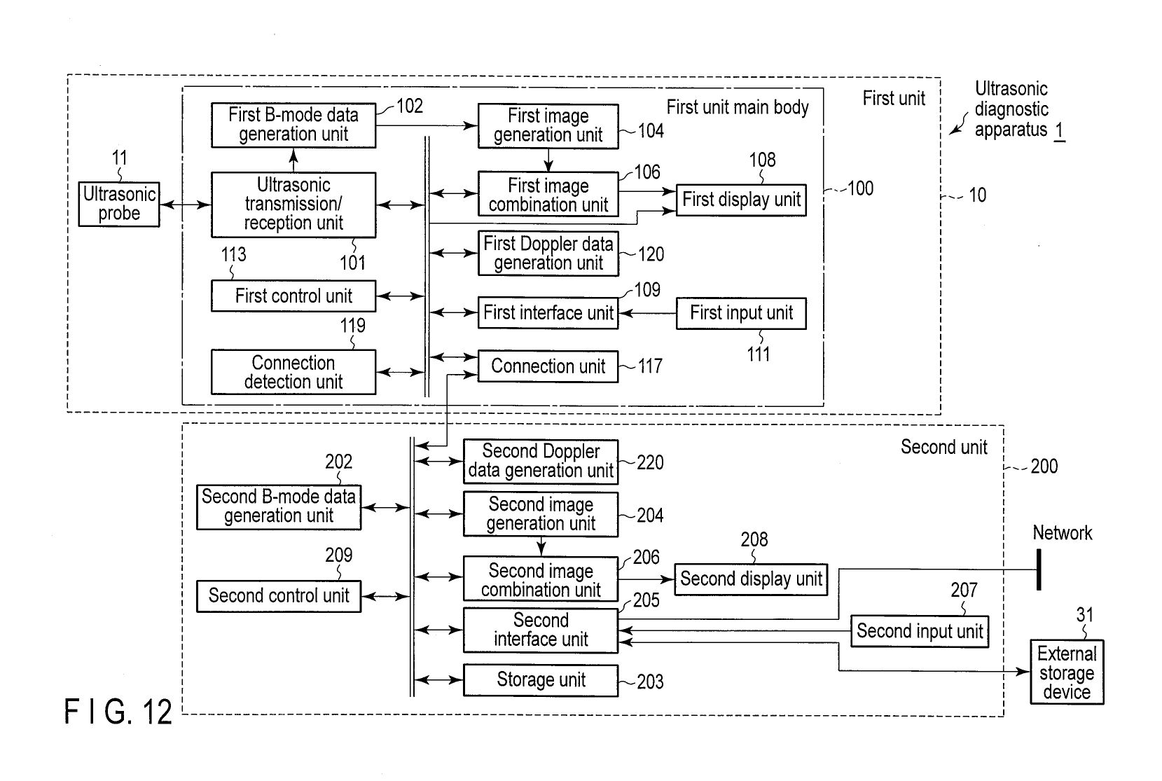

FIG. 12 is a block diagram showing the arrangement of an ultrasonic diagnostic apparatus 1 according to the seventh embodiment.

A first Doppler data generation unit 120 generates the first Doppler data based on a reception signal. The first Doppler data generation unit 120 has a processing function and operation speed which are minimum necessary for the generation of the first Doppler data.

A first image generation unit 104 generates the first Doppler image based on the first Doppler data. The first image generation unit 104 has a processing function and operation speed which are minimum necessary for the generation of the first Doppler image. The generated first Doppler image has image quality (resolution), field angle, and the like which are minimum necessary for an ultrasonic image.

A first control unit 113 controls an ultrasonic transmission/reception unit 101 to obtain a reception signal associated with the generation of the first B-mode data. More specifically, the first control unit 113 controls the ultrasonic transmission/reception unit 101 to apply a driving voltage to each of a plurality of piezoelectric transducers located near the center of the opening of an ultrasonic probe 11. That is, the ultrasonic transmission/reception unit 101 applies a driving voltage to each of some of the piezoelectric transducers of the ultrasonic probe 11 under the control of the first control unit 113. The scanning range is limited under the control of the first control unit 113.

When a second unit 200 is connected to a first unit 10 via a connection unit 117, the first control unit 113 releases the control from each unit mounted in a first unit main body 100. That is, the first control unit 113 releases the control right of each unit except for a first B-mode data generation unit 102, a first Doppler data generation unit 120, and the first image generation unit 104. When the first unit 10 is connected to the second unit 200, the control right of the first ultrasonic image generation unit may be moved to a second control unit 209.

The second control unit 209 includes a digital signal processor (to be referred to as a DSP hereinafter) having a higher operation speed than the DSP mounted in the first control unit 113. When the second unit 200 is connected to the first unit 10 via the connection unit 117, the second control unit 209 controls the ultrasonic transmission/reception unit 101 as well as controlling the respective units of the second unit 200. More specifically, the second control unit 209 controls the ultrasonic transmission/reception unit 101 to apply driving voltages to piezoelectric transducers larger in number than those to which driving voltages are applied in the first unit 10 alone. This increases the number of channels driven and improves the S/N ratio.

Note that the second control unit 209 may control a second image generation unit 204 to execute various types of real-time clinical application processing. Clinical applications include, for example, an application for blood flow parameter analysis (specifying arterial bloods, analyzing luminance changes due to ultrasonic bubbles, and the like). If, for example, an elastic image includes the heart of an object, the second control unit 209 may have a function associated with cardiac function analysis. The functions associated with cardiac function analysis are, for example, a function of analyzing two-dimensional or three-dimensional myocardial strain (the movement of a cardiac chamber wall or the like).

A second Doppler data generation unit 220 generates the second Doppler data larger in data amount than the first Doppler data based on a reception signal. The operation speed of the second Doppler data generation unit 220 is higher than that of the first Doppler data generation unit 120. This improves the throughput of a flow processor per unit time. The first Doppler data generation unit 120 is included in the second ultrasonic image generation unit.

The second image generation unit 204 generates the second Doppler image having a higher resolution than the first Doppler image based on the second Doppler data. Note that the second image generation unit 204 generates a rendering image based on the second Doppler data. At this time, the second image generation unit 204 has a rendering function. The second image generation unit 204 is equipped with a graphics processing unit (to be referred to as a GPU hereinafter) higher in operation speed than the GPU mounted in the first image generation unit 104. The second image generation unit 204 can generate an elastic image (elastography) based on the second Doppler data.

According to the above arrangement, the following effects can be obtained.

The ultrasonic diagnostic apparatus 1 is configured to be separated into the first unit 10 having basic performance and function associated with the generation of Doppler images and the second unit 200 which has higher function and performance than the basic function and performance and generate Doppler images. With this arrangement, when the first unit 10 is connected to the second unit 200, the second ultrasonic image generation unit generates the second Doppler image instead of the first ultrasonic image generation unit. This improves the performance and function as compared with when the first unit 10 is used alone. In addition, this embodiment allows the expansion of a clinical application. As described above, connecting the first unit 10 to the second unit 200 will improve the throughput and expand the function.

In addition, it is possible to reduce the power consumption of the first unit 10 of the ultrasonic diagnostic apparatus 1. This improves the operating time of the first unit 10 of the ultrasonic diagnostic apparatus 1. In addition, this can simplify the arrangement of the first unit 10 of the ultrasonic diagnostic apparatus 1, leading to a reduction in the size of the first unit 10 of the ultrasonic diagnostic apparatus 1. Furthermore such reductions in power consumption and size can improve the operating time and portability. Therefore, this improves the convenience of the ultrasonic diagnostic apparatus 1 in home care, sports activities, disaster sites, and the like.

In addition, connecting the first unit 10 to the second unit 200 makes it possible to use the ultrasonic diagnostic apparatus 1 as a floor-standing type ultrasonic diagnostic apparatus higher in function than the portable type ultrasonic diagnostic apparatus in a hospital or the like. In addition, the ultrasonic diagnostic apparatus 1 can lead to a reduction in cost as compared with a case in which both a floor-standing type ultrasonic diagnostic apparatus and a portable type ultrasonic diagnostic apparatus are purchased. As described above, the ultrasonic diagnostic apparatus 1 can provide ultrasonic diagnostic services in accordance with conditions around objects and diagnostic purposes at low cost and with high efficiency.

In addition, each function according to each embodiment can be implemented by installing programs for executing the processing in a computer such as a workstation and expanding them in the memory. In this case, the programs which can cause the computer to execute the corresponding techniques can be distributed by being stored in storage media such as magnetic disks (Floppy® disks, hard disks, and the like), optical disks (CD-ROMs, DVDs, and the like), and semiconductor memories.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasonic diagnostic apparatus comprising a first unit including an ultrasonic probe and a second unit configured to be detachably connected to the first unit, wherein
the first unit comprises
a transmission/reception unit configured to transmit/receive an ultrasonic wave to/from an object through the ultrasonic probe and generate a reception signal,
a first ultrasonic image generation unit configured to generate a first ultrasonic image at a first processing speed and with a first processing function based on the reception signal,
a connection unit configured to connect the first unit to the second unit, and
a connection detection unit configured to detect connection between the first unit and the second unit, and
the second unit comprises
a second ultrasonic image generation unit configured at a second processing speed faster than the first processing speed and with a second processing function higher than the first processing function, the second ultrasonic image generation unit configured to generate a second ultrasonic image based on the reception signal upon detection of the connection, the second ultrasonic image having larger data than the first ultrasonic image.

2. The apparatus of claim 1, wherein
the first unit comprises a first control unit configured to control the transmission/reception unit and the first ultrasonic image generation unit and to decontrol the transmission/reception unit and the first ultrasonic image generation unit upon the detection of the connection, and
the second unit comprises a second control unit configured to control the transmission/reception unit and the second ultrasonic image generation unit upon the detection of the connection.

3. The apparatus of claim 2, wherein
the first ultrasonic image generation unit comprises
a first B-mode data generation unit configured to generate first B-mode data based on the reception signal, and
a first image generation unit configured to generate the first ultrasonic image at the first processing speed and with the first processing function based on the first B-mode data, and
the second ultrasonic image generation unit comprises
a second B-mode data generation unit configured to generate second B-mode data having larger data than the first B-mode data based on the reception signal, and
a second image generation unit configured to generate the second ultrasonic image at the second processing speed and with the second processing function based on the second B-mode data.

4. The apparatus of claim 3, wherein the second ultrasonic image has a higher resolution than the first ultrasonic image.

5. The apparatus of claim 2, wherein
the first ultrasonic image generation unit comprises
a first Doppler data generation unit configured to generate first Doppler data based on the reception signal, and
a first image generation unit configured to generate the first ultrasonic image at the first processing speed and with the first processing function based on the first Doppler data, and
the second ultrasonic image generation unit comprises
a second Doppler data generation unit configured to generate second Doppler data having larger data than the first Doppler data based on the reception signal, and
a second image generation unit configured to generate the second ultrasonic image at the second processing speed and with the second processing function based on the second Doppler data.

6. The apparatus of claim 2, wherein
the first control unit is configured to control the transmission/reception unit to drive part of a plurality of transducers provided on the ultrasonic probe, and
the second control unit is configured to control the transmission/reception unit to drive the plurality of transducers.

7. An ultrasonic diagnostic apparatus comprising at least one first unit including an ultrasonic probe and a second unit configured to be connected to the first unit via a network, wherein
the first unit comprises
a transmission/reception unit configured to transmit/receive an ultrasonic wave to/from an object through the ultrasonic probe and generate a reception signal,
a first data transfer unit configured to transfer the reception signal to the second unit via the network, and
a display unit configured to display an ultrasonic image associated with the object, and
the second unit comprises
a B-mode data generation unit configured to generate B-mode data based on the transferred reception signal,
a Doppler data generation unit configured to generate Doppler data based on the transferred reception signal,
an image generation unit configured to generate the ultrasonic image based on at least one of the B-mode data and the Doppler data, and
a second data transfer unit configured to transfer the generated ultrasonic image to the first unit, and
wherein the display unit is configured to display the ultrasonic image transferred by the second data transfer unit.

8. An ultrasonic diagnostic apparatus comprising at least one first unit including an ultrasonic probe and a second unit configured to be connected to the first unit via a network, wherein
the first unit comprises
a transmission/reception unit configured to transmit/receive an ultrasonic wave to/from an object through the ultrasonic probe and generate a reception signal,
a B-mode data generation unit configured to generate B-mode data based on the reception signal,
a Doppler data generation unit configured to generate Doppler data based on the reception signal, and
a first data transfer unit configured to transfer the generated B-mode data and the generated Doppler data to the second unit via the network, and
the second unit comprises
an image generation unit configured to generate an ultrasonic image based on at least one of the B-mode data and the Doppler data, and
a second data transfer unit configured to transfer the ultrasonic image to the first unit via the network.

9. The apparatus of claim 8, wherein the first unit comprises a display unit configured to display the ultrasonic image transferred from the second unit.

10. An ultrasonic diagnostic apparatus comprising at least one first unit including an ultrasonic probe and a second unit configured to be connected to the first unit via a network, wherein
the first unit comprises
a transmission/reception unit configured to transmit/receive an ultrasonic wave to/from an object through the ultrasonic probe and generate a reception signal,
a first ultrasonic image generation unit configured to generate a first ultrasonic image at a first processing speed and with a first processing function based on the reception signal, and
a first data transfer unit configured to transfer the reception signal to the second unit via the network, and
the second unit comprises
a second ultrasonic image generation unit configured at a second processing speed faster than the first processing speed and with a second processing function higher than the first processing function, the second ultrasonic image generation unit configured to generate a second ultrasonic image based on the reception signal, the second ultrasonic image having larger data than the first ultrasonic image.

11. The apparatus of claim 10, wherein
the first unit comprises
a connection detection unit configured to detect connection between the first unit and the second unit via the network, and
a first control unit configured to control the transmission/reception unit and the first ultrasonic image generation unit and to decontrol the transmission/reception unit and the first ultrasonic image generation unit upon detection of the connection, and
the second unit comprises
a second control unit configured to control the transmission/reception unit and the second ultrasonic image generation unit upon the detection of the connection.
